(19) United States
(12) Patent Application Publication
(10) Pub. No.: US 2010/0291205 A1
(43) Pub. Date: Nov. 18, 2010

(54) PHARMACEUTICAL COMPOSITIONS AND METHODS FOR MITIGATING RISK OF ALCOHOL INDUCED DOSE DUMPING OR DRUG ABUSE

(75) Inventors: Ken Downie, Livingston (GB); Anders Vagno Pedersen, Virum (DK); Anne-Mette Haahr, Birkerod (DK); Pernille Hoyrup Hemmingsen, Bagsvaerd (DK); Daniel Bar-Shalom, Kokkedal (DK)

Correspondence Address: STOEL RIVES LLP - SLC, 201 SOUTH MAIN STREET, SUITE 1100, ONE UTAH CENTER, SALT LAKE CITY, UT, 84111, US

(73) Assignee: EGALET A/S, Vaerlose (DK)

(21) Appl. No.: 12/523,045

(22) PCT Filed: Jan. 16, 2008
(86) PCT No.: PCT/DK08/00016

§ 371 Date: Nov. 19, 2009

Related U.S. Application Data
(60) Provisional application No. 60/885,106, filed on Jan. 16, 2007.

(30) Foreign Application Priority Data
Jan. 16, 2007 (DK) PA 2007 00063

(51) Int. Cl. A61K 9/52; A61K 9/00; A61K 9/22; A61P 25/04; A61K 31/485

(52) U.S. Cl. 424/457; 424/486; 424/468; 514/282; 424/484

(57) ABSTRACT

Abuse resistant polyglycol-based pharmaceutical compositions are disclosed. The composition contains one or more polyglycols and one or more active substances and it is resistant to crushing, melting and/or extraction. Moreover, such compositions have the same or lower solubility in ethanolic-aqueous medium, i.e. they are not subject to ethanol-induced dose dumping effect.

### FIELD OF THE INVENTION

The present invention relates to the use of a composition that has such a composition and structure that there is no substantial risk of drug abuse by using the methods generally applied by drug addicts. Moreover, such compositions have proved to mitigate the risk of alcohol induced dose dumping. The composition comprises one or more polyglycols (notably one or more homo- or copolymers). Moreover, the compositions have been manufactured by heating in order to melt or soften the polymer followed by solidification. The compositions are notably in the form of oral solid dosage forms.

### BACKGROUND OF THE INVENTION

### Drug Abuse

FDA is in general aware of problems related to abuse of narcotic drugs. In the present context, the term “abuse” is intended to denote the use of a drug in order to induce euphoria, i.e. the use is not intended to cure a disease or alleviate disease symptoms, but rather for obtaining intoxication. In order to abuse a drug, the active drug substance is normally isolated and/or dissolved (in more or less pure form), i.e. it involves a deliberately attempt to isolate or dissolve the active drug substance from the composition in order to obtain the active drug substance in a more concentrated form so that the active drug substance can be injected or otherwise taken to obtain an intoxication effect.

As an example FDA had approved OxyContin (a narcotic drug) for treatment of moderate to severe pain. The active ingredient in OxyContin is oxycodone HCl (hydrochloride) with an addiction potential similar to that of morphine. It has been reported that a widespread abuse and misuse of OxyContin occurs as it is possible to crush the controlled-release capsule and then make an intravenous injection or snorting. These possible events have lead to a high level of abuse (Meyer, R. J., 2005).

WO 2006/058249 (Acura Pharmaceuticals) relates to methods and compositions for deterring abuse of orally administered pharmaceutical products. The abuse-resistance is obtained by use of a combination of a gel-forming polymer, a nasal mucosal irritating surfactant and a flushing agent. The compositions can be crushed (cf. page 7, line 19) and thus be available for inhalation, but the inclusion of a nasal mucosal irritating agent and/or a flushing agent probably limits such a use. The present applicant has repeated Examples 45 and 46 of WO 2006/058249, wherein the compositions include use of, a polyglycol, and the results confirm that the compositions can be crushed.

WO 2006/106344 (MW Encap Limited) relates to abuse resistant capsules. The capsules include a modifier that has a high melting point therefore melt at a temperature too high to inject. An example using poloxamer 188 is given, but the present Applicant has shown that such a composition can be crushed, i.e. it may be subject for sniffing, or dissolved, i.e. it may be subject for injection or to drink.

### Dose Dumping

Unintended, rapid drug release in a short period of time of the entire amount or a significant fraction of the active drug substance retained in a controlled release dosage form is often referred to as “dose dumping”. It has been reported that some modified-release oral dosage forms contain active drug substances and excipients that exhibit higher solubility in ethanolic solutions compared to water. Such products can be expected to exhibit a more rapid drug dissolution and release rate in the presence of ethanol. Therefore, in theory, concomitant consumption of alcoholic beverages along with these products might be expected to have the potential to induce dose dumping (FDA's ACPS Meeting 2005).

As an example FDA approved Palladone (hydromorphone hydrochloride extended-release) in September 2004. Palladone was capsules for treatment of persistent, moderate to severe pain. Shortly after approval it was reported that “dose-dumping” occurred in patients if Palladone was taken along with alcohol. Even low concentrations of alcohol showed serious effects on the release of hydromorphone from Palladone. In July 2005, FDA suspended sales and marketing of the drug because of the potential for severe side effects if the drug is taken together with alcohol (Meyer, R. J., 2005).

The potential risk of dose dumping has only recently attracted attention in regulatory approval procedures. A regulatory approach to evaluate the potential for alcohol dose dumping is being developed. The goal of the regulatory approach should be to minimize the risk of alcohol induced dose dumping from controlled release forms, irrespective of any warnings on product labelling and instructions by health care providers.

US 2006/0193912 (Penwest Pharmaceuticals) describe compositions that are expected to exhibit reduced alcohol induced dose dumping. The compositions include a mixture of gums and ionizable gel strength enhancing agent.

US 2003/0118641 (Boehringer Ingelheim Corporation) relates to abuse-resistant sustained-release opioid formulations. The compositions include an ionic exchange resin and a matrix-forming polymer. Moreover, the compositions include specific types of opioid compounds, which have been found to possess high resistance to extraction with common solvents. In contrast, the present invention does not include ionic exchange resin.

To the best of the inventors' knowledge no composition has been developed that is generally applicable to a wide range of active drug substance while at the same time, the risk for alcohol induced dose dumping is reduced or even eliminated.

Accordingly, there is a need for developing pharmaceutical composition that have reduced risk for drug abuse and/or that releases the active drug substance independent on presence of any alcohol (e.g. ethanol).

### DETAILED DESCRIPTION OF THE INVENTION

The present invention is based on controlled release compositions containing a matrix composition comprising a) polymer or a mixture of polymers, b) an active drug substance and optionally c) one or more pharmaceutically acceptable excipients that is without alcohol induced dose dumping and have excellent properties with respect to avoiding drug abuse. The matrix composition according to the invention has (in total) a lower (or equal) solubility and/or release rate in alcohol containing media (e.g. ethanol) than in aqueous media (e.g. water, buffer). As demonstrated in the examples herein, the alcohol containing medium may include e.g. ethanol in a wide concentration range. Thus, the medium may contain from about 2.5 to about 80% v/v of alcohol, the remaining part being a water-based medium (i.e. water or aqueous buffer).

The active drug substance in the above mention matrix composition optionally comprising chosen polymers and excipients in a suitable ratio attains an unchanged or lower dissolution rate when tested in alcohol containing media as compared to aqueous media.

Accordingly, the present invention is a further development of the Applicant's earlier development of controlled release compositions containing a matrix composition comprising a polymer that is a substantially water soluble polyglycol (WO 89/009066, WO 90/004015, WO 95/022962, WO 99/051208, WO 03/024426, WO 03/024429, WO 03/024430, WO 04/084868, WO 04/041252, WO 04/084869, WO 06/128471). The Applicant has surprisingly found that such a composition has excellent properties with respect to avoiding alcohol induced dose dumping as well as avoiding drug abuse. Accordingly, in one aspect the present invention relates to a novel use of a known composition. Some of the compositions may of course be novel and in such cases the invention relates to such novel compositions as well.

The controlled release compositions that are suitable for use according to the invention are normally intended for oral administration. Thus, they are solid oral dosage form such as single unit dosage form, but may also be present as individual units in the form of a multiple unit composition, where each of the individual units has the desired properties.

More specifically, the invention relates to the use of i) a polymer and ii) an active drug substance for the preparation of pharmaceutical composition that mitigates or is without alcohol induced dose dumping. Typically, the solubility or release rate of the composition is lower or substantially the same in alcohol than that in water. More specifically, the solubility or release is equal or at least 1.25 times lower such as at least 1.5 times lower, at least 2 times lower in alcohol than in water, notably 5 times, 10 times, 25 times, 50 times or 100 times lower.

In a specific embodiment the polymer comprises a polyglycol, notably a substantially water soluble, crystalline and semi-crystalline polyglycol.

An important property of the polymer is that it is thermoplastic.

In order to ensure that the use of a composition mitigates alcohol induced dose dumping, the ratio (R₅₀) between t_{50% w/w}(40% w/w ethanol in medium 1) and t_{50% w/w}(medium 1) is 1 or more. t_{50% w/w}(medium 1) denotes the time it takes to release 50% w/w of the active drug substance from the pharmaceutical composition in an in vitro dissolution test according to USP 30, NF 25, (711), Apparatus 2, paddle employing water optionally buffered to a specific pH as dissolution medium (medium 1), and t_{50% w/w}(40% w/w ethanol in medium 1) denotes the time it takes to release 50% w/w of the active drug substance from the pharmaceutical composition in an in vitro dissolution test according to USP 30, NF 25, (711), Apparatus 2, paddle employing 40% w/w ethanol in medium 1 as dissolution medium.

In a specific embodiment, the ratio R₅₀ is at the most 5 such as at the most 4, at the most 3 or at the most 2. Notably, the ratio R₅₀ is from 1 to 1.5 such as, e.g., from 1 to 1.4, from 1 to 1.3, from 1 to 1.2, from 1 to 1.1, from 1 to 1.05, or about 1.

The same may also apply for ratios determined e.g. when 25%, 30%, 40%, 60%, 70%, 80%, 90% and/or 95% w/w has been released, the conditions being as described above.

As mentioned above, alcohol induced dose dumping is an overlooked problem especially for controlled release composition that normally contains a larger dose of the active drug substance than e.g. a plain tablet. The larger dose is due to the fact that the controlled release composition is designed to be effective for an extended period of time and, accordingly, a larger dose is normally required. Other kinds of alcohol induced effects have been subject to investigations, including changes in pharmacokinetic behavior of the active drug substance (e.g. altered clearance) and pharmacodynamic interactions (e.g. effects on the central nervous system), but only recently alcohol induced dose dumping with respect to alteration of the release of the active drug substance from the composition has become an issue. To this end, it has been suggested that an easy manner to investigate whether a composition potentially will be subject to alcohol induced dose dumping is to subject the composition to an dissolution test using a dissolution medium with and without alcohol and investigate whether there are any differences in the release pattern under the two different conditions. The harshest conditions are in a dissolution medium containing 40% (v/v) ethanol. If the dissolution rate of the composition is substantially unaffected or slower then it is likely to assume that no alcohol induced dose dumping will take place in vivo.

As seen from the examples herein, a composition of the type disclosed herein (i.e. based on the Applicant's earlier developments, see WO 89/009066, WO 90/004015, WO 95/022962, WO 99/051208, WO 03/024426, WO 03/024429, WO 03/024430, WO 04/084868, WO 04/041252, WO 04/084869, WO 06/128471, which is hereby incorporated by reference) fulfils the above-mentioned requirement and therefore, it is contemplated that no alcohol induced dose dumping will take place in vivo after oral administration of the compositions. In fact, the dissolution rate of the active drug substance from a composition as described herein is unchanged or lower when tested in alcohol-containing medium as compared to an aqueous medium. Accordingly, the present invention provides a general solution to avoiding alcohol induced dose dumping while at the same time providing a composition with well-documented controlled release pattern (e.g. zero order) in an extended period of time.

A composition for use according to the present invention has solubility in water that is higher than that in ethanol. The solubility rate and the dissolution rate are both highly dependent on solubility. The general solubility equation (GSE) has shown to be excellent for a qualitative description of the solubility-behavior of any given substance. The GSE states that the solubility of a substance in water depends upon the melting entropy, the temperature difference between the melting point of the substance and the operating temperature, and the hydrophobicity of the substance expressed as the octanol-water partition coefficient. In this particular case, the ethanol-water partition coefficient is that of most interest. The hydrophobicity of the substance is estimated by the functional groups comprising the substance. Functional groups consisting of electronegative atoms (e.g. N, O, P, S etc.) prone to form hydrogen bonds are by definition hydrophilic and hence water-soluble. On the other hand substances containing aromatic rings and/or long carbon-chains are more lipophilic, hence less water-soluble. Although ethanol is capable of forming hydrogen bonds, a hydrophilic substance will be less soluble in ethanol compared to water, and a lipophillic substance will be more soluble in ethanol compared to water. The substance is e.g. active drug substance, polymer (polyglycol) and pharmaceutically acceptable excipients.

In the case of a matrix composition containing polymers, active drug substance and possible excipients, the composition may be designed to possess a ratio of hydrophilic/hydrophobic groups that facilitates a lower solubility in ethanol.

The solubility rate of a given matrix composition depends on other parameters, besides the solubility, the melting temperature and the hydrophobicity of the matrix composition, the pH environment, the ion strength etc. The gel-layer thickness, the swelling rate (wetability) and the strength of the gel-layer (the rate of polymer-disentanglement) also have an impact on the rate of the erosion process. As shown herein, the ratio of hydrophilic/hydrophobic functionally groups in the matrix composition are the determining factor that leads to a lower solubility rate in ethanol-containing solutions.

Moreover, for hydrophilic active drug substances, freely soluble in aqueous media and alcohol containing media, the dissolution rate may be lower when tested in an alcohol containing media than in an aqueous media. For hydrophobic active drug substances practically insoluble in aqueous media and with higher solubility in alcohol containing media, the dissolution rate may be unaffected when tested in aqueous and alcohol containing media, respectively.

### Drug Abuse

In another aspect, the invention relates to the use of i) a polymer comprising a polyglycol and ii) a active drug substance for the preparation of pharmaceutical composition as defined herein, the composition being resistant to isolate and/or dissolve the active drug substance from the composition by crushing, melting and/or ethanol extraction, whereby the composition is resistant to drug abuse. A composition that can fulfil all there requirements below without any specific addition of e.g. irritating agents, flushing agent, emetic agent, stool softeners, opioid antagonist or bad-tasting or bad-flavouring agent is desired and such compositions are provided herein.

The likelihood of a composition being subject to drug abuse can be tested by three different tests:

1. Crushing test
2. Melting test

### 3. Extraction/dissolving

In the crushing test, the composition is subjected to crushing using a hammer or an apparatus designed to measure the hardness of an oral dosage form. A suitable apparatus is specified in Ph.Eur or in the Examples herein. If the composition disintegrates into particles, then it may be possible to dissolve or suspend these particles and use them for abuse purposes. Moreover, if it is possible to disintegrate (crunch) the composition, then it is possible to use the powder for snorting or sniffing and in this way abuse the composition, however, if it is not possible to crush the composition in this test, then there will be no particles to use for such abuse purposes.

In the melting test, the composition is subjected to heating e.g. on a spoon or by exposure to microwave induced heating. If the composition is suitable for abuse purposes, the composition should become so liquid that it is possible to inject it without being too hot. However, if this is not the case, the composition is not suitable for abuse purposes.

In the extraction test it is tested whether it is possible to extract the active drug substance from the composition by means of normally available organic solvents. If it is possible to dissolve the composition then if may be possible to misuse the drug. On the contrary, if it is not possible, then it is likely that the composition cannot be misused.

As demonstrated in the examples herein, a composition as described herein the outcome of all the three tests describe above indicate that the composition is not likely to be misused.

The present invention is based on controlled release compositions comprising a) polymer or a mixture of polymers, more specifically a polyglycol, b) an active drug substance and optionally c) one or more pharmaceutically acceptable excipients. In the following paragraphs polymers and pharmaceutically acceptable excipients suitable for use in such a composition as well as relevant active drug substances are described. The composition is designed for oral administration and, normally, it is designed for controlled release. However, with respect to the drug abuse aspect, the composition need not be a controlled release composition as the abuse aspect is of relevance for immediate as well as controlled release compositions.

The composition is of such nature that it is basically impossible to abuse either by crushing, melting, extraction, dissolving or similar. Furthermore, the composition exhibits decreased (or essentially the same) release rate in alcohol containing media as compared to a purely aqueous media. The release rate from the composition will depend on several parameters such as in an unlimited list: solubility of the polymer, active drug substance and the excipients, the wetability of the composition, the diffusion of water into the composition, the enthalpy of melting and enthalpy of solubilization, and the disentanglement rate of the polymer during dissolution.

Controlled release dosage forms are used to extend the release from the dosage form for an extended period of time. In the present context the term “controlled release” is used to designate a release a desired rate during a predetermined release period. Terms like “modified”, “delayed”, “sustained”, “prolonged”, “extended” etc. release are in the present context synonyms to the term “controlled release”.

The controlled release may be obtained by means of several mechanisms. In particular the release may be governed by the mechanism of dissolving/solubilization of the active drug substance, by the mechanism of diffusion controlled release (e.g. diffusion coating and/or matrix formulation), by the mechanism of pH triggered release such as for example enteric coatings, by the mechanism of osmotic pump, by the mechanism of ion exchange or by the mechanism of biodegradation. The mechanism of release may also be a combination of the above mentioned mechanisms.

In a specific embodiment of the invention the release mechanism is primarily erosion from a composition composed matrix of a polymer, an active drug substance and excipients. The mechanism of erosion enables the composition to release with a rate depending on the exposed area. In particular the release is zero order when the matrix is partly covered by a non-erodible and non-permeable coat in a cylindrical shape exposing the matrix in the two ends of composition.

The design of the pharmaceutical composition is based on the finding that it is possible to control the release from such a composition by ensuring that the release predominantly takes place by erosion. In order to ensure erosion based release a balance must be obtained between the diffusion rate of water into the matrix composition and the dissolution rate of the matrix composition

A composition according to the invention containing an active drug substance is typically for oral administration and may be in the form of multiple unit (e.g. pellets or mini tablets) and single unit dosages form e.g. in the form of tablets, capsules or sachets etc. Due to the possibility of controlling the release rate of the active drug substance the composition may be adapted for oral administration 1-6 times a day, normally 1-4 times daily such as 1-3 times, 1-2 times or 1 times daily. The technology may also provide compositions for administration only once or twice daily. In the present context the term “once daily” is intended to mean that it is only necessary to administer the pharmaceutical composition once a day in order to obtain a suitable therapeutic and/or prophylactic response.

The dosage of the active drug substance depends on the particular substance, the age, weight-condition etc. of the human that will be treated with the composition etc. All such factors are well known to a person skilled in the art.

The rate at which the active drug substance is released from the matrix is a predetermined rate, i.e. a rate, which is controllable over a certain period of time. The release rate required in each particular instance may inter alia depend on the amount of active drug substance to be released for it to exert the desired effect, as well as on the overall dosage of the active drug substance contained in the matrix.

In a specific embodiment the controlled release of the active drug substance obtainable from the pharmaceutical composition of the invention, it is possible to obtain a substantially constant rate of release of the active substance over a specific period of time, corresponding to the dosage necessary for the treatment in question, so that adherence to a strict dosage regimen, e.g. requiring administration of a active drug substance at set intervals up to several times a day, may be dispensed with.

Furthermore, it is possible to include two or more different active drug substances in the pharmaceutical composition of the invention, and the two or more different active drug substances may be adapted to be released at different concentrations and/or intervals, thus making it easier for patients to follow a prescribed regimen.

An additional advantage of a pharmaceutical composition of the invention, compared to other known controlled release compositions, is that it may be produced by relatively simple and inexpensive methods.

The pharmaceutical composition according to the invention may furthermore be used in the preparation of a multiple units composition, e.g. in the form of a capsule, sachets or tablet. A multiple-units composition is a composition, which comprises a multiplicity of individual units in such a form that the individual units will be made available upon disintegration of the composition in the stomach and or intestine of humans. Thus, in this case, at least some of the individual units in said multiple unit composition will consist of the composition of the invention, the individual units being of a size, which allows them to be incorporated into such a composition.

### Polymers

Suitable polymers for use according to the invention typically comprises a polyglycol, e.g. in the form of a homopolymer and/or a copolymer. In a specific embodiment the polymer is substantially water soluble, thermoplastic, crystalline, semi-crystalline or amorphous or a mixture of substantially water soluble, crystalline, semi-crystalline or amorphous polymers. Suitable polymers for use in a composition according to the invention are polyethylene glycols, including derivatives such as mono and dimethoxypolyethylene glycols (mPEGs) polyethylene oxides and/or block copolymers of ethylene oxide and propylene oxide.

Polyethylene glycols (PEGs) are linear polydisperse polymers composed of repeating units of ethylene glycol. Their chemical formula is HOCH₂[CH₂OCH₂]ₘCH₂OH where m represents the average number of repeating units. Alternatively, the general formula H[OCH₂CH₂]ₙOH may be used to represent polyethylene glycol, where n is a number m in the previous formula+1. See the structural presentations of polyethylene glycol below. n is the average number of oxyethylene groups. n equals m+1.

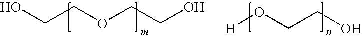

Polyethylene oxides (PEOs) are linear polydisperse nonionic polymers composed of repeating units of ethylene oxide. Their chemical formula is HO[CH₂CH₂O]ₙH where n represents the average number of oxyethylene groups. See the structural presentation of polyethylene oxide below. n is the average number of oxyethylene groups. Depending on preparation method high molecular weigh PEO may have one terminal methyl group.

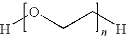

Polyethylene glycols are mixtures of addition of ethylene glycol. In general PEG refers to polymers chains with molecular weights below 20,000, while PEO refers to higher molecular weights polymers. However, because of the similarities between PEO and PEG, the terms are often used interchangeably for the same compound.

Polyethylene glycols and/or polyethylene oxides, which are suitable for use in the matrix composition are those having a molecular weights of from about 20,000 daltons, such as, e.g., from about 20,000 to about 700,000 daltons, from about 20,000 to about 600,000 daltons, from about 35,000 to about 500,000 daltons, from about 35,000 to about 400,000 daltons, from about 35,000 to about 300,000 daltons, from about 50,000 to about 300,000 daltons, such as, e.g. about 35,000 daltons, about 50,000 daltons, about 75,000 daltons, about 100,000 daltons, about 150,000 daltons, about 200,000 daltons, about 250,000 daltons, about 300,000 daltons or about 400,000 daltons.

In a specific embodiment the polymer is a polyethylene oxide or a polyethylene glycol that has a molecular weight of about 20,000 daltons, about 35,000 daltons, about 50,000 daltons, about 100,000 daltons, about 200,000 daltons, about 300,000 daltons and about 400,000 daltons. PEG is commercially available with average molecular weights up to 35 000. PEO is commercially available with average molecular weights up to 8,000,000. In specific embodiment, the polymer is a PEO having a molecular weight of at least about 100,000 such as, e.g., from about 100,000 to about 8,000,000, from about 100,000 to about 7,000,000, from about 100,000 to about 5,000,000, from about 100,000 to about 4,000,000, from about 100,000 to about 2,000,000, from about 100,000 to about 1,000,000, form about 100,000 to about 900,000. When PEO is employed with a molecular weight in the tower end, the PEO typically has a molecular weight as mentioned in the preceding paragraph. Commercially available PEOs with a molecular weight in the higher end have typically the following molecular weights: about 900,000, about 1,000,000, about 2,000,000, about 4,000,000, about 5,000,000, about 7,000,000, about 8,000,000.

Poloxamers are copolymers or block copolymers and are a range of non-ionic surfactants of polyethylene glycol (PEG) and polypropylene glycol (PPG).

In chemical abstracts Diol EO/PO block copolymers are described under the scientific name -hydroxy-hydroxypoly(oxyethylene)poly(oxypropylene)-poly(oxyethylene)-block copolymer in combination with the CAS register number.

In specific embodiments a suitable poloxamer for use in a composition of the invention has a HLB value of at least about 18 such as, e.g., at least about 20. The mean molecular weight of a suitable poloxamer is typically at least about 2,000.

Typical block copolymers of ethylene oxide and propylene oxide have a molecular weight of from about 2,000 daltons, typically about 3,000 to about 30,000 daltons such as, e.g. from about 4,000 to about 15,000 daltons. If the copolymer is the sole thermoplastic polymer present in the composition it must not bee too brittle in order to avoid abuse by crushing of the composition, i.e. it must have an HLB value of about 18 to about 24.

Mixtures of PEO with different average molecular weights can be used in order to obtain a PEO with a desirable average molecular weight. The same applies to PEG.

The polymer has a melting point higher than the body temperature of the human in which the composition is to be used. Thus, the polymer(s) employed in the matrix composition will suitably have a melting point of about 20-120° C. such as, e.g. from about 30 to about 100° C. or from about 40 to about 80° C.

In addition to a polymer of a polyglycol type as described above other polymers may be suitable for use in a pharmaceutical composition provided that the solubility and/or release rate of the active substance from the composition in water is higher than or equal to the solubility of the matrix in 40% w/w ethanol in water. Thus, in other embodiments of the invention, the polymer or an additional polymer to the polyglycol may be selected from one or more of the following polymers: modified or unmodified water soluble natural polymers such as glucomannan, galactan, glucan, polygalacturonic acid, polyxylane, polygalactomannans, rhanogalacturonan, polyxyloglycan, arabinogalactan, and starch, cellulose, chitosan, alginate, fibrin, collagen, gelatin, hyaluronic acid, amylopectin, pectin including low methylated or methoxylated pectins, dextran and fatty acids and alcohols; synthetic polymers such as polyvinylpyrrolidone (PVP), PVA, PVB, Eudragit L methyl ester, Eudragit L, Eudragit RL, Eudragit E, Eudragit S, PHPV, PHA, PCL, PLGA and PLA; and hydrogels made from the polymers or combined polymers mentioned above and or from polymers originated from: HEMA, HEEMA, MEMA, MEEMA, EDGMA, NVP, VAc, AA, acrylamide, MAA, HPMA, PEGA, PEGMA, PEGDMA, PEGDA, and PEGDMA.

One or more polymers are typically present in a composition of the invention in a concentration amount of from 5 to 99.9% w/w such as from 10 to 95% such as from 15% to 90%, such as from 20 to 85%, such as from 30% to 85% calculated as w/w % of the composition.

In those cases, where mixture of polymers are present in the composition, the concentration of an individual polymer in the composition may typically be from about 0% to about 95% w/w such as, e.g., from about 10% to about 90% w/w, from about 10% to about 80% w/w, from about 10% to about 70% w/w, from about 10% to about 60%, from about 10% to about 50%, from about 15% to about 50% w/w, from about 15% to about 45% w/w, from about 15% to about 40% w/w, from about 20% to about 40% w/w, from about 20% to about 35% w/w or from about 20% to about 30% w/w.

The total concentration of the polymers (notably the sum of homo- and copolymers of the polyglycol type) in the composition is typically from about 5 to about 99.9% w/w such as from about 10 to about 95% w/w, from about 15% to about 90% w/w, such as from 20 to 85%, such as from 30% to 85% from about 30 to about 99% w/w such as, e.g., from about 35 to about 95% w/w, from about 35 to about 90% w/w, from about 35 to about 85% w/w, from about 35 to about 80% w/w, from about 40 to about 75% w/w, from about 45 to about 70% w/w, from about 45 to about 65% w/w. from about 55 to about 85% w/w or from about 60 to about 85% w/w.

The concentration of the polyglycol homopolymer is typically from about 5 to about 99.9% w/w such as from about 20 to about 90% w/w, from about 30 to about 90% w/w, and, in those cases where the homopolymer is the only thermoplastic polymer present in the composition, then the concentration is normally from about 50 to about 95% w/w such as, e.g. from about 55 to about 90% w/w, from about 60 to about 90%, from about 65 to about 90%, from about 70% to about 90% or from about 70 to about 85% w/w.

The concentration of the polyglycol copolymer, if present in combination with a polyglycol homopolymer, is typically from about 1 to about 60% w/w such as, e.g. from about 2.5 to about 50% w/w, from about 5 to about 45% w/w. If the copolymer is the sole thermoplastic polymer in the composition the concentration may be from about 5 to about 99.5% w/w such as those ranges described above and described for the homopolymer.

Active Drug Substances with Abuse Potential or Safety Risk

An active drug substance in a composition for use according to the invention is a therapeutically, prophylactically and/or diagnostically active drug substance (herein also abbreviated “active drug substance”). In principle, the use of a composition to avoid alcohol dose dumping can be of relevance for any active drug substance.

However, the main interest is with respect to active drug substances with abuse potential or safety risk.

Examples of active drug substance with abuse potential or safety risk suitable for use according to the present invention are:

1-(1-Phenylcyclohexyl)pyrrolidine, 1-(2-Phenylethyl)-4-phenyl-4-acetoxypiperidine, 1-[1-(2-Thienyl)-cyclohexyl]piperidine, 1-[1-(2-Thienyl)cyclohexyl]pyrrolidine, 1-Methyl-4-phenyl-4-propionoxy-piperidine, 1-Phenylcyclohexylamine, 1-Piperidinocyclohexanecarbonitrile, 2,5-Dimethoxy-4-ethylamphetamine, 2,5-Dimethoxyamphetamine, 2C-B-(4-bromo-2,5-dimethoxypenethylamine), 2C-D (2,5-dimethoxy-4-methylphenethylamine), 2C-I (4-iodo-2,5-dimethoxy-phenethylamine), 2C-T-2 (2,5-dimethoxy-4-ethylthiophenethylamine), 2C-T-4 (2,5-dimethoxy-4-isopropyl thiophenethylamine), 2C-T-7 (2,5-dimethoxy-4-(n)-propylthiopenethylamine), 3,4-Methylene-dioxymethamphetamine, 3,4,5-Trimethoxyamphetamine, 3,4-Methylenedioxyamphetamine, 3,4-Methylenedioxy-N-ethylamphetamine, 3-Methylfentanyl, 3-Methylthiofentanyl, 4-Bromo-2,5-dimethoxyamphetamine, 4-Bromo-2,5-dimethoxyphenethylamine, 4-Methoxyamphetamine, 4-Methyl-2,5-dimethoxyamphetamine, 4-Methylaminorex (cis isomer), 5-MeO-DMT (5-Methoxy-N,N-diisopropyltryptamine), 5-MeO-DMT (5-Methoxy-N,N-dimethyltryptamine), 5-Methoxy-3,4-methylenedioxyamphetamine, Acetorphin, Acetorphine, Acetyl-alpha-methylfentanyl, Acetyl-alpha-methylfentanyl, Acetyldihydrocodeine, Acetylmethadol, Acetylmethadol, Alfentanil, Allobarbital, Allylprodin, Allylprodine, Alphacetylmethadol except levo-alphacetylmethadol, Alpha-ethyltryptamine, Alphameprodine, Alphamethadol, Alphamethadol, Alpha-Methylfentanyl, Alpha-Methylthiofentanyl, Alphaprodine, Alprazolam, Amfepramon, Amfetaminil, Amineptin, A minorex, Amobarbital, Amphetamine, AmyInitrit (all isomers of the amyl group), Anabolic steroids, Anileridine, Aprobarbital, Barbital, Barbituric acid derivative, BDB (3,4-methylenedioxyphenyl)-2-butanamine), Benzethidin, Benzethidine, Benzoylecgonine, Benzphetamine, Benzphetamine, Benzylmethylketon, Benzylmorphine, Betacetylmethadol, Beta-Hydroxy-3-methylfentanyl, Beta-Hydroxyfentanyl, Betameprodine, Betameprodine, Betamethadol, Betaprodine, Bezitramide, Bezitramide, Boldenone, Brolamfetamin, Bromazepam, Brotizolam, Bufotenine, Buprenorphine, Butabarbital, Butalbital, Butobarbital, Butorphanol, BZP (A 2)(1-benzylpiperazin), Camazepam, Cannabis, Carfentanil, Catha edulis, Cathine, Cathinone, Chloral betaine, Chloral hydrate, Chlordiazepoxide, Chlorhexadol, Chlorotestosterone (same as clostebol), Chlorphentermine, Clobazam, Clonazepam, Clonitazene, Clonitazene, Clorazepate, Clortermine, Clostebol, Clotiazepam, Cloxazolam, Coca Leaves, Cocaine, Codeine, Codeine & isoquinoline alkaloid, Codeine methylbromide, Codeine-N-oxide, Codoxim, Cyclobarbital (Hexemal NFN), Cyprenorphine, Dehydrochlormethyltestosterone, Delorazepam, Desomorphine, Dexamfetamine, Dexfenfluramine, Dextromoramide, Dextropropoxyphene, Diacetylmorphine, Diampromide, Diazepam, Dichloralphenazone, Diethylpropion, Diethylthiambutene, Diethyltryptamine, Difenoxin, Dihydrocodeine, Dihydroetorphine, Dihydromorphine, Dihydrotestosterone, Dimenoxadol, Dimepheptanol, Dimethylthiambutene, Dimethyltryptamine, Dioxaphetyl butyrate, Diphenoxylate, Dipipanone, Diprenorphine, Dronabinol, Drostanolone, Drotebanol, Ecgonine, Estazolam, Ethchlorvynol, Ethinamate, Ethyl loflazepate, Ethylestrenol, Ethylmethylthiambutene, Ethylmorphine, Ethylmorphine, Eticyclidin, Etilamfetamine, Etonitazene, Etorphine, Etoxeridine, Etryptamine, Fencamfamin, Fenethylline, Fenetylline, Fenfluramine, Fenproporex, Fentanyl, Fludiazepam, Flunitrazepam, Fluoxymesterone, Flurazepam, Formebolone, Fungi and Spores of the sepcies Psilocype Semilanceata, Furethidine, Gammahydroxybutanic acid, Glutethimide, Halazepam, Haloxazolam, Heroine, Hydrocodone, Hydrocodone & isoquinoline alkaloid, Hydromorphinol, Hydromorphone, Hydroxypethidine, Ibogaine, Isobutylnitrit, Isomethadone, Ketamine, Ketazolam, Ketobemidone, Levamfetamine, Levo-alphacetylmethadol, Levo-methamphetamine, Levomethorphan, Levomoramide, Levophenacylmorphan, Levorphanol, Loprazolam, Lorazepam, Lormetazepam, Lysergic acid, Lysergic acid amide, Lysergic acid diethylamide, Marijuana, Mazindol, MBDN(N-methyl-1-(3,4-methylenedioxyphenyl)-2-butanamine), mCPP (1-(3-chlorphenyl)piperazine), Mebutamate, Mecloqualone, Medazepam, Mefenorex, MeOPP (1-(4-methoxyphenyl)piperazine), Meperidine, Meperidine intermediate, Meprobamate, Mescaline, Mesocarb, Mesterolone, Metamfetamine, Metazocine, Methadone, Methadone intermediate, Methamphetamine, Methandienone, Methandranone, Methandriol, Methandrostenolone, Methaqualone, Methcathinone, Methenolone, Methohexital, Methyldesorphine, Methyldihydromorphine, Methylphenidate, Methylphenobarbital (mephobarbital), Methyltestosterone, Methyprylone, Metopone, Mibolerone, Midazolam, Modafinil, Moramide-intermediate, Morpheridine, Morphine, Morphine methylbromide, Morphine methylsulfonate, Morphine-N-oxide, Myrophine, N,N-Dimethylamphetamine, Nabilone, Nalorphine, Nandrolone, N-Ethyl-1-phenylcyclohexylamine, N-Ethyl-3-piperidyl benzilate, N-Ethylamphetamine, N-Hydroxy-3,4-methylenedioxyamphetamine, Nicocodeine, Nicocodine, Nicodicodine, Nicomorphine, Nimetazepam, Nitrazepam, N-Methyl-3-piperidyl benzilate, Noracymethadol, Norcodeine, Nordiazepam, Norethandrolone, Norlevorphanol, Normethadone, Normorphine, Norpipanone, Norpipanone, Opium, Oxandrolone, Oxazepam, Oxazolam, Oxycodone, Oxymesterone, Oxymetholone, Oxymorphone, Para-Fluorofentanyl, Parahexyl, Paraldehyde, Pemoline, Pentazocine, Pentobarbital, Petrichloral, Peyote, Phenadoxone, Phenampromide, Phenazocine, Phencyclidine, Phendimetrazine, Phenmetrazine, Phenobarbital, Phenomorphan, Phenoperidine, Phentermine, Phenylacetone, Pholcodine, Piminodine, Pinazepam, Pipradrole, Piritramide, PMMA (paramethyxymethyl amphetamine), Prazepam, Proheptazine, Properidine, Propiram, Psilocybine, Psilocyn, Pyrovalerone, Quazepam, Racemethorphane, Racemoramide, Racemorphane, Remifentanil, Salvia divinorum, Salvinorin A, Secobarbital, Secobarbital, Sibutramine, SPA, Stanolone, Stanozolol, Sufentanil, Sulfondiethylmethane, Sulfonethylmethane, Sulfonmethane, Talbutal, Temazepam, Tenamfetamin, Testolactone, Testosterone, Tetrahydrocannabinols, Tetrazepam, TFMPP (1-(3-triflourmethylphenyl)piperazine), Thebacon, Thebaine, Thiamylal, Thiofentanyl, Thiopental, Tiletamine & Zolazepam in Combination, Tilidine, Trenbolone, Triazolam, Trimeperidine, Vinbarbital, Zaleplon, Zipeprol, Zolpidem, Zopiclon.

Other suitable examples include alfentanil, allylprodine, alphaprodine, aniloridine, benzylmorphine, bezitramide, buprenorphine, butophanol, clonitazene, codeine, cyclazocine, desomorphine, dextromoramide, dezocine, diapromide, dihydrocodeine, dihydromorphine, dimenoxadol, dimephetanol, dimethylthiambutene, dioxaphetyl butyrate, dipipanone, eptazocine, ethoheptazine, ethylmethylthiambutene, ethylmorphine, etonitazene, fentanyl, heroin, hydrocodone, hydromorphone, hydroxypethidine, isomethadone, dextropropoxyphene, ketobemidone, levallorphan, levorphanol, levophenacylmorphan, lofentanil, meperidine, meptazinol, metazocine, methadone, metopon, morphine, morphine 6-glucuronide, morphine 3-glucuronide, myrophine, nalbuphine, narccine, nicomorphine, norlevorphanol, normethadone, nalorphine, normorphine, norpipanone, opium, oxycodone, oxycodeine, oxymorphone, papavereturn, pentazocine, phenadoxone, phenomorphan, phenazocine, phenoperidine, piminodine, piritramide, propheptazine, promedol, properidine, propiram, propoxyphene, sufentanil, tilidine, tramadol, thebaine, levo-alphacetylmethadol (LAAM), remifentanil, carfentanyl, ohmefentanyl, MPPP, prodine, PEPAP, levomethorphan, etorphine, lefetamine, loperamide, diphenoxylate, pethidine.

Other suitable examples also include Anabolic steroids, cannabis, cocaine and diazepam.

All of the above mentioned active drug substances may also be in the form of pharmaceutically acceptable salts, complexes, solvates or anhydrates thereof, and, if relevant, isomers, enantiomers, racemic mixtures, and mixtures thereof.

In specific embodiments, the active drug substance is buprenorphine, codeine, dextromoramide, dihydrocodeine, fentanyl, hydrocodone, hydromorphone, morphine, pentazocine, oxycodeine, oxycodone, oxymorphone and tramadol.

The term “pharmaceutically acceptable salts” of an active drug substance includes alkali metal salts such as, e.g., sodium or potassium salts, alkaline earth metal salts such as, e.g., calcium and magnesium salts, and salts with organic or inorganic acid like e.g. hydrochloric acid, hydrobromic acid, nitric acid, sulfuric acid, phosphoric acid, citric acid, formic acid, maleic acid, succinic acid, tartaric acid, methansulphonic acid, toluenesulphonic acid etc.

The term “solvates” includes hydrates or solvates wherein other solvates than water are involved such as, e.g., organic solvents like chloroform and the like.

Furthermore, the active drug substance may be in any of its crystalline, polymorphous, semi-crystalline, amorphous or polyamorphous forms.

The concentration of the active drug substance in a composition for use according to the invention depends on the specific active drug substance, the disease to be treated, the condition of the patient, the age and gender of the patient etc. The above-mentioned active drug substances are well-known active drug substances and a person skilled in the art will be able to find information as to the dosage of each active drug substance and, accordingly, he will know how to determine the amount of each active drug substance in a composition.

The active drug substance is typically present in a composition of the invention in a concentration amount of from 0.01-99% w/w such as, e.g., from about 0.01% to about 90% w/w, from about 0.01% to about 80% w/w, from about 0.01% to about 70% w/w, from about 0.01% to about 50% w/w or from about 0.01% to about 40% w/w.

### Pharmaceutically Acceptable Excipients

The composition may also contain other excipients as well, e.g. in order to improve the technical properties of the matrix composition so that it may be easier to produce or in order to improve the properties of the composition such as release rate of the active drug substance, stability of the active drug substance or of the composition itself etc.

A suitable pharmaceutically acceptable excipient for use in a matrix composition of the invention may be selected from the group consisting of fillers, diluents, disintegrants, glidants, pH-adjusting agents, viscosity adjusting agents, solubility increasing or decreasing agents, osmotically active agents and solvents.

Suitable excipients include conventional tablet or capsule excipients. These excipients may be, for example, diluents such as dicalcium phosphate, calcium sulfate, lactose or sucrose or other disaccharides, cellulose, cellulose derivatives, kaolin, mannitol, dry starch, glucose or other monosaccharides, dextrin or other polysaccharides, sorbitol, inositol or mixtures thereof; binders such as alginic acid, calcium alginate, sodium alginate, starch, gelatin, saccharides (including glucose, sucrose, dextrose and lactose), molasses, panwar gum, ghat gum, mucilage of isapol husk, carboxymethylcellulose, methylcellulose, veegum, larch arabolactan, polyethylene glycols, ethylcellulose, water, alcohols, waxes, polyvinylpyrrolidone such as PVP K90 or mixtures thereof; lubricants such as talc, silicium dioxide, magnesium stearate, calcium stearate, stearic acid, hydrogenated vegetable oils, sodium benzoate, sodium chloride, leucine, carbowax 4000, magnesium lauryl sulfate, Sodium laurilsulfate, Stearyl alcohol, Polysorbate 20, Polysorbate 60, Polysorbate 80, Macrogol stearate, Macrogol lauryl ether, Stearoyl macrogolglycerides, Sorbitan stearate, Sorbitan laurate, Macrogol glycerol hydroxystearat, colloidal silicon dioxide and mixtures thereof, disintegrants such as starches, clays, cellulose derivatives including crosscarmellose, gums, aligns, various combinations of hydrogencarbonates with weak acids (e.g. sodium hydrogencarbonate/tartaric acid or citric acid) crosprovidone, sodium starch glycolate, agar, cation exchange resins, citrus pulp, veegum, glycollate, natural sponge, bentonite, sucralfate, calcium hydroxyl-apatite or mixtures thereof.

Furthermore, the composition may comprise one or more agents selected from the group consisting of sweetening agents, flavouring agents and colouring agents, in order to provide an elegant and palatable preparation. Examples are maltol, citric acid, water soluble FD&C dyes and mixtures thereof with corresponding lakes and direct compression sugars such as Di-Pac from Amstar. In addition, coloured dye migration inhibitors such as tragacanth, acacia or attapulgite talc may be added. Specific examples include Calcium carbonate, 1,3,5-trihydroxybenzene, Chromium-cobalt-aluminium oxide, ferric ferrocyanide, Ferric oxide, Iron ammonium citrate, Iron (III) oxide hydrated, Iron oxides, Carmine red, Magnesium carbonate and Titanium dioxide.

Plasticizer may be incorporated in the composition. A suitable plasticizer is selected from such as e.g. mono- and di-acetylated monoglycerides, diacetylated monoglycerides, acetylated hydrogenated cottonseed glyceride, glyceryl cocoate, Polyethylene glycols or polyethylene oxides (e.g. with a molecular weight of about 1,000-500,000 daltons), dipropylene glycol salicylate glycerin, fatty acids and esters, phthalate esters, phosphate esters, amides, diocyl phthalate, phthalyl glycolate, mineral oils, hydrogenated vegetable oils, vegetable oils, acetylated hydrogenated soybean oil glycerides, Castor oil, acetyl tributyl citrate, acetyl triethyl citrate, methyl abietate, nitrobenzene, carbon disulfide, β-naphtyl salicylate, sorbitol, sorbitol glyceryl tricitrate, fatty alcohols, cetostearyl alcohol, cetyl alcohol, stearyl alcohol, oleyl alcohol, myristyl alcohol, sucrose octaacetate, alfa-tocopheryl polyethylene glycol succinate (TPGS), tocopheryl derivative, diacetylated monoglycerides, diethylene glycol monostearate, ethylene glycol monostearate, glyceryl monooleate, glyceryl monostearate, propylene glycol monostearate, macrogol esters, macrogol stearate 400, macrogol stearate 2000, polyoxyethylene 50 stearate, macrogol ethers, cetomacrogol 1000, lauromacrogols, nonoxinols, octocinols, tyloxapol, poloxamers, polyvinyl alcohols, polysorbate 20, polysorbate 40, polysorbate 60, polysorbate 65, polysorbate 80, polysorbate 85, sorbitan monolaurate, sorbitan monooleate, sorbitan monopalmitate, sorbitan monostearate, sorbitan sesquioleate, sorbitan trioleate, sorbitan tristearate and sucrose esters, amyl oleate, butyl oleate, butyl stearate, diethylene glycol monolaurate, glycerol tributyrate, Cumar W-1, Cumar MH-1, Cumar V-1, Flexol B-400, monomeric polyethylene ester, Piccolastic A-5, Piccalastic A-25, Beckolin, Clorafin 40, acetyl tributyl citrate, acetyl triethyl citrate, benzyl benzoate, butoxyethyl stearate, butyl and glycol esters of fatty acids, butyl diglycol carbonate, butyl ricinoleate, butyl phthalyl butyl glycolate, camphor, dibutyl sebacate, dibutyl tartrate, diphenyl oxide, glycerine, HB-40, hydrogenated methyl ester of rosin, methoxyethyl oleate, monoamylphthalate, Nevillac 10, Paracril 26, technical hydroabietyl alcohol, triethylene glycol dipelargonate, solid aliphatic alcohols and mixtures thereof.

Preferred stabilizers (chemical) include TPG e.g. in the form of TPGS due to surfactant properties, BHA, BHT, t-butyl hydroquinone, calcium ascorbate, gallic acid, hydroquinone, maltol, octyl gallate, sodium bisulfite, sodium metabisulfite, tocopherol and derivates thereof, citric acid, tartaric acid, and ascorbic acid. Other stabilisers include trivalent phosphorous like e.g phosphite, phenolic antioxidants, hydroxylamines, lactones such as substituted benzofuranones. Hindered phenols, thiosynergists and/or hindered amines, acids (ascorbic acid, erythorbic acid, etidronic acid, hypophosphorous acid, nordihydroguaiaretic acid, propionic acid etc.), phenols, dodecyl gallate, octyl gallate, 1,3,5-trihydroxybenzene, organic and inorganic salts (calcium ascorbate, sodium ascorbate, sodium bisulphite, sodium metabisulfite, sodium sulfite, potassium bisulphite, potassium metabisulphite), esters (calcium ascorbate, dilauryl thiodipropionate, dimyristyl thiodipropionate, distearyl thiodipropionate), pyranon (maltol), and vitamin E (tocopherol, D-α-tocopherol, DL-α-tocopherol, tocopheryl acetate, d-α-tocopheryl acetate, dl-α-tocopheryl acetate. However, other anti-oxidative agents known in the art may be used according to the present invention. Other suitable stabilizer is selected from such as e.g. sorbitol glyceryl tricitrate, sucrose octaacetate.

Modifier may be incorporated in the composition. A suitable modifier is selected from such as e.g. fatty acids and esters, fatty alcohols, cetyl alcohol, stearyl alcohol, mineral oils, hydrogenated vegetable oils, vegetable oils, acetylated hydrogenated soybean oil glycerides, Castor oil, phosphate esters, amides, phthalate esters, glyceryl cocoate oleyl alcohol, myristyl alcohol, sucrose octaacetate, diacetylated monoglycerides, diethylene glycol monostearate, ethylene glycol monostearate, glyceryl monooleate, glyceryl monostearate, propylene glycol monostearate, macrogol esters, macrogol stearate 400, macrogol stearate 2000, polyoxyethylene 50 stearate, macrogol ethers, cetomacrogol 1000, lauromacrogols, poloxamers, polyvinyl alcohols, sorbitan monolaurate, sorbitan monooleate, sorbitan monopalmitate, sorbitan monostearate, sorbitan sesquioleate, sorbitan trioleate, sorbitan tristearate, ethylcellulose, cellulose acetate, cellulose propionate, cellulose nitrate, cellulose derivative selected from the group consisting of methylcellulose, carboxymethylcellulose and salts thereof, cellulose acetate phthalate, microcrystalline cellulose, ethylhydroxyethylcellulose, ethylmethylcellulose, hydroxyethylcellulose, hydroxyethylmethylcellulose, hydroxypropylcellulose, hydroxypropylmethylcellulose, hydroxymethylcellulose and hydroxymethylpropylcellulose, cellulose acetate, polylactic acid or polyglycolic acid and copolymers thereof, methacrylates, a co-polymer of methacrylate-galactomannan etc., Polyvinyl alcohols, glycerinated gelatin, cocoa butter.

Other suitable modifier may be selected from the group consisting of inorganic acids, inorganic bases, inorganic salts, organic acids or bases and pharmaceutically acceptable salts thereof, saccharides, oligosaccharides, polysaccharides, polyethylene glycol derivatives and cellulose and cellulose derivatives.

Alternatively or additionally, a suitable pharmaceutically acceptable excipient is a mono-, di-, oligo, polycarboxylic acid or amino acids such as, e.g. acetic acid, succinic acid, citric acid, tartaric acid, acrylic acid, benzoic acid, malic acid, maleic acid, sorbic acid etc., aspartic acid, glutamic acid etc.

Examples of suitable organic acids include acetic acid/ethanoic acid, adipic acid, angelic acid, ascorbic acid/vitamin C, carbamic acid, cinnamic acid, citramalic acid, formic acid, fumaric acid, gallic acid, gentisic acid, glutaconic acid, glutaric acid, glyceric acid, glycolic acid, glyoxylic acid, lactic acid, levulinic acid, malonic acid, mandelic acid, oxalic acid, oxamic acid, pimelic acid, and pyruvic acid.

Examples of suitable inorganic acids include pyrophosphoric, glycerophosphoric, phosphoric such as ortho and meta phosphoric, boric acid, hydrochloric acid, and sulfuric acid.

Examples of suitable inorganic compounds include aluminium.

Examples of organic bases are p-nitrophenol, succinimide, benzenesulfonamide, 2-hydroxy-2cyclohexenone, imidazole, pyrrole, diethanolamine, ethyleneamine, tris (hydroxymethyl) aminomethane, hydroxylamine and derivates of amines, sodium citrate, aniline, hydrazine.

Examples of inorganic bases include aluminium oxide such as, e.g., aluminium oxide trihydrate, alumina, sodium hydroxide, potassium hydroxide, calcium carbonate, ammonium carbonate, ammonium hydroxide, KOH and the like.

Suitable pharmaceutically acceptable salts of an organic acid is e.g. an alkali metal salt or an alkaline earth metal salt such as, e.g. sodium phosphate, sodium dihydrogenphosphate, disodium hydrogenphosphate etc., potassium phosphate, potassium dihydrogenphosphate, potassium hydrogenphosphate etc., calcium phosphate, dicalcium phosphate etc., sodium sulfate, potassium sulfate, calcium sulfate, sodium carbonate, sodium hydrogencarbonate, potassium carbonate, potassium hydrogencarbonate, calcium carbonate, magnesium carbonate etc., sodium acetate, potassium acetate, calcium acetate, sodium succinate, potassium succinate, calcium succinate, sodium citrate, potassium citrate, calcium citrate, sodium tartrate, potassium tartrate, calcium tartrate etc.

A suitable inorganic salt for use in a matrix composition of the invention is sodium chloride, potassium chloride, calcium chloride, magnesium chloride etc.

Saccharides such as glucose, ribose, arabinose, xylose, lyxose, xylol, allose, altrose, inosito, glucose, sorbitol, mannose, gulose, Glycerol, idose, galactose, talose, mannitol, erythritol, ribitol, xylitol, maltitol, isomalt, lactitol, sucrose, fructose, lactose, dextrin, dextran, amylose, xylan.

Polyethylene glycol derivatives such as e.g. polyethylene glycol di(2-ethyl hexoate), polyethylene glycols (200-600 daltons) or polyethylene oxides, e.g. with a molecular weight of about 800-500,000 daltons, typically about 1,000-100,000 daltons, more typically 1,000-50,000 daltons, especially about 1,000-10,000 daltons, in particular about 1,500-5,000 daltons, and mixtures thereof.

Cellulose and cellulose derivative selected from the group consisting of methylcellulose, carboxymethylcellulose and salts thereof, microcrystalline cellulose, ethylhydroxyethylcellulose, ethylcellulose, cellulose acetate, cellulose proprionate, cellulose nitrate, cellulose acetate phthalate, ethylmethylcellulose, hydroxyethylcellulose, hydroxyethylmethylcellulose, hydroxypropylcellulose, hydroxypropylmethylcellulose, hydroxymethylcellulose and hydroxymethylpropylcellulose.

### Preparation

The delivery system as well as the composition of the invention may be produced by various methods which are either known per se in the pharmaceutical industry or which, for example, are used in the production of polymer-based materials, depending upon the desired embodiment and the materials employed in the composition in question. One advantage of the composition according to the invention is that it may be produced by methods, which are relatively simple and inexpensive.

Suitable preparation methods for compositions according to the invention include extrusion, injection molding, tabletting, capsule filling, thermoforming, spray coating, micro encapsulation and other methods of preparing controlled release compositions.

The controlled release composition may be prepared by several different methods. Many systems for controlled release are marketed and it is currently an aim for the industry to reduce the risk of dose dumping, drug abuse or alcohol induced dose dumping in each of the systems.

In other words, in addition to a less frequent administration, the real challenge in controlled release delivery may be expressed by the goal of decreasing the incidence of adverse effects and at the same time increasing the effect of the treatment. This can only be obtained by an interaction between the specific pharmacological properties of the active drug substance and the composition.

High concentrations or a fast rise in the concentration of for example morphine is one important factor resulting in side effects including the risk of getting addicted to morphine. The fear of addiction is often a major obstacle for initiation of the otherwise effective pain treatment with morphine both in the view of the clinical personnel as well as in the view of the patients themselves.

Compositions for controlled release according to the invention may be prepared in numerous ways giving rise to different release mechanisms. Particularly the composition may be prepared by 1, 2 or multiple component injection moldings, by conventional tablet compression, by micro encapsulation, by 1, 2 or multiple component extrusions, by capsule filling or by thermoforming. In cases were a preparation is needed in order to make the controlled release properties before/after the above mentions preparation steps, the preparation may also comprise separate steps as for example wet granulation, dry granulation, melt granulation, pelletizing, spray coating, electrostatic coating or other forms of controlled release forming preparation methods.

In a particular example the composition is prepared by two component injection molding of a matrix and a coat partly covering the matrix and exposing two ends of the composition for erosion governed release.

A composition may also be produced by, for example, injection moulding, co-extrusion of the coating with the matrix composition and the active drug substance, extrusion and dip coating, injection moulding and dip coating, or by extrusion or injection moulding and solvent coating by spraying or dipping. Multiple component injection moulding, or a combination of these methods.

### Geometry

The release mechanisms described above depends on the geometry of the composition. For example erosion based release from a matrix depends on the exposed area of the matrix. In this case the area may be manipulated by employment of a coat that is not subject to erosion and thus covering the areas of the matrix that hence will not be a releasing site. In particular, a cylindrical composition with the two ends exposing the eroding matrix will give rise to zero order release because the releasing area is constant.

The geometric form of the composition is very important for the obtainment of the above-mentioned controlled release. Thus, in one embodiment of the invention, the pharmaceutical composition has a geometric shape, which enables a substantially constant surface area to become exposed during erosion of the matrix.

In a specific example, the compositions employed are coated in such a manner that the surface has a substantially constant or controlled surface area during release or erosion. In the present context controlled surface area relates to a predetermined surface area typically predicted from the shape of the coat of the unit dosage system. It may have a simple uniform cylindrical shape or the cylindrical form can have one or more tapered ends in order to decrease (or increase) the initial release period.

As another example, in diffusion based systems the release will furthermore depend on the thickness of the diffusion layer and in this case the release will depend both on the diffusion area and thickness of the diffusion system.

As yet another example the release mechanism of dissolving/solubilization also depend on the releasing area and the release rate may be controlled by covering parts of the releasing matrix by a coat. Controlling the coverage of the matrix by the coat hence refers to covering from 0 to 100% of the matrix by a coat.

### Coating

The composition may be partly or fully covered by a coat with specific properties in such a way that the exposed area of the matrix may be controlled by the use of a coat. In a specific example the coat is substantially insoluble, non-erodable and non-permeable to water leaving only the exposed areas of the matrix for release. Such a coat may be composed of a cellulose derivative which has thermoplastic properties, plasticizer or plasticizers and/other functional excipients.

The coating may also be a coating, which is substantially soluble in and permeable to fluids such as body fluids during the intended release period provided that the coating dissolves so much slower than the matrix composition that the coating remains intact until the matrix has eroded and/or released the active drug substance. Examples of suitable polymers include polyglycols as described herein.

The coating may further comprise any of the below-mentioned matrix materials in a form, which erodes at a substantially slower rate than the rest of the matrix. The coating may thus comprise a matrix of one or more substantially water soluble crystalline polymers and, optionally, a non-ionic emulsifier, the coating being one which is eroded in the aqueous phase at a substantially slower rate than the matrix composition comprising the active drug substance, whereby a substantially controlled area of the matrix composition comprising the active drug substance is exposed during erosion and/or release of the matrix composition, and whereby the coating is substantially eroded upon erosion and/or release of the matrix composition comprising the active drug substance. Such a coating will be designed so that its longitudinal erosion rate is substantially the same as the longitudinal erosion and/or release rate of the matrix, whereby the matrix and the coating will erode longitudinally towards the centre of the composition at substantially the same rate. Thus, when the matrix composition has been completely eroded and/or released by the aqueous medium, the coating will also be substantially completely eroded. A matrix composition having such a coating has the obvious advantage of being completely biodegraded upon release of the active drug substance.

In an embodiment of the invention, the coating is one, which biodegrades, disintegrates, crumbles or dissolve after erosion of the matrix and/or during the release of the active drug substance. A coating applied for an erosion matrix will remain intact as long as it is supported by the matrix containing the active drug substance, but it lacks the ability to remain intact after erosion of the matrix, because it then biodegrades, disintegrates or crumbles, so that it will not remain in e.g. a human for any significant amount of time after the complete erosion of the matrix and the release of the active drug substance.

In an interesting embodiment, the controlled release composition of the invention further comprises a coating having at least one opening exposing at least one surface of the matrix, the coating being one which crumbles and/or erodes upon exposure to the aqueous medium at a rate which is equal to or slower than the rate at which the matrix erodes in the aqueous medium, allowing exposure of said surface of the matrix to the aqueous medium to be controlled.

Polymers useful as coatings are such as e.g. cellulose derivative e.g. ethylcellulose, cellulose acetate, cellulose propionate, cellulose nitrate, cellulose derivative selected from the group consisting of methylcellulose, carboxymethylcellulose and salts thereof, cellulose acetate phthalate, microcrystalline cellulose, ethylhydroxyethylcellulose, ethylmethylcellulose, hydroxyethylcellulose, hydroxyethylmethylcellulose, hydroxypropylcellulose, hydroxypropylmethylcellulose, hydroxymethylcellulose and hydroxymethylpropylcellulose, cellulose acetate, Eudragit L methyl ester, Eudragit RL, Eudragit E, polyamide, polyethylene, polyethylene terephthalate, polypropylenem polyurethane, polyvinyl acetate, polyvinyl chloride, silicone rubber, latex, polyhydroxybutyrate, polyhydroxyvalerate, teflon, polylactic acid or polyglycolic acid and copolymers thereof, copolymers such as ethylene vinyl acetate (EVA), styrene-butadienestyrene (SBS) and styrene-isoprene-styrene (SIS). The coating may also be copolymers of polylactic acid and polyglycolic acid. The coating may also be an enteric coating employing methacrylates Eudragit L, Eudragit S, Eudragit FS, a co-polymer of methacrylate-galactomannan etc.

The invention is further illustrated in the non-limiting examples with the appended drawings in which

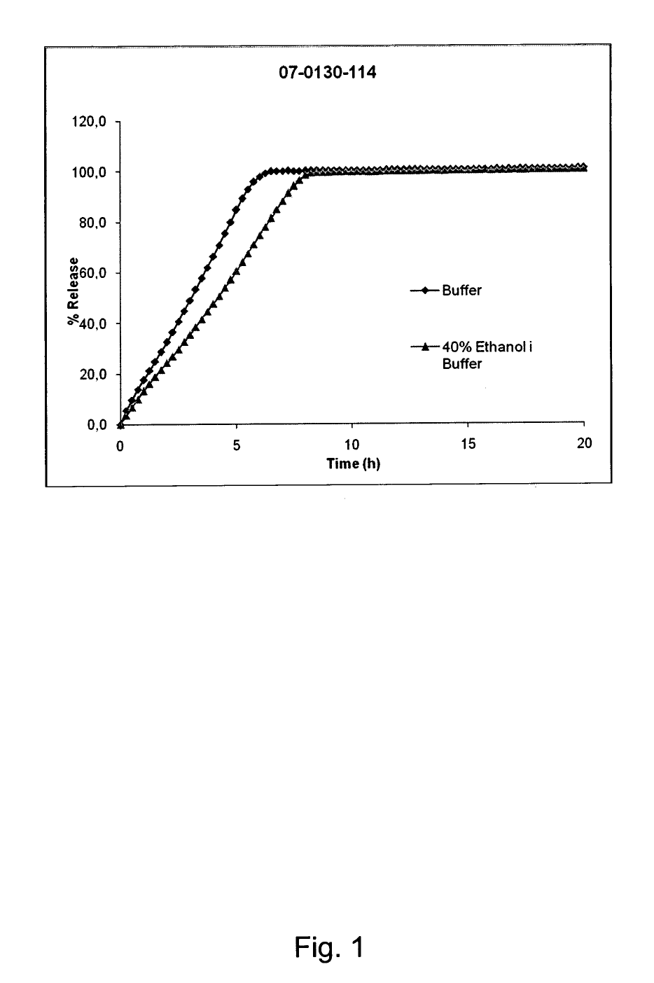

FIG. 1 shows in vitro dissolution of Oxycodone hydrochloride, batch No. 07-0130-114, from Example 1

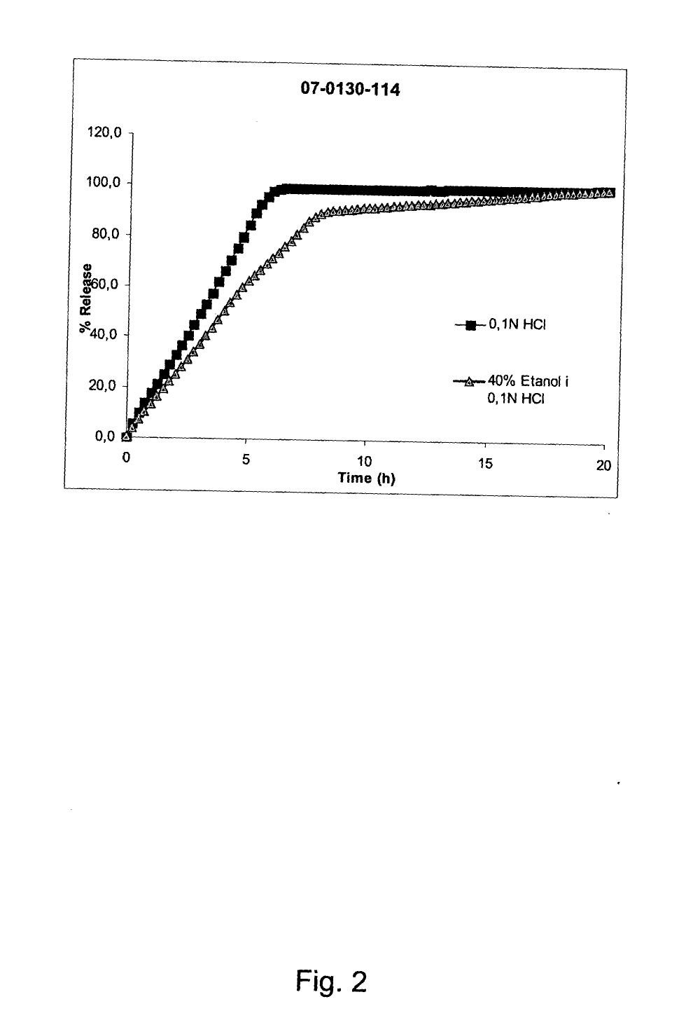

FIG. 2 shows in vitro dissolution of Oxycodone hydrochloride, batch No. 07-0130-114, from Example 1

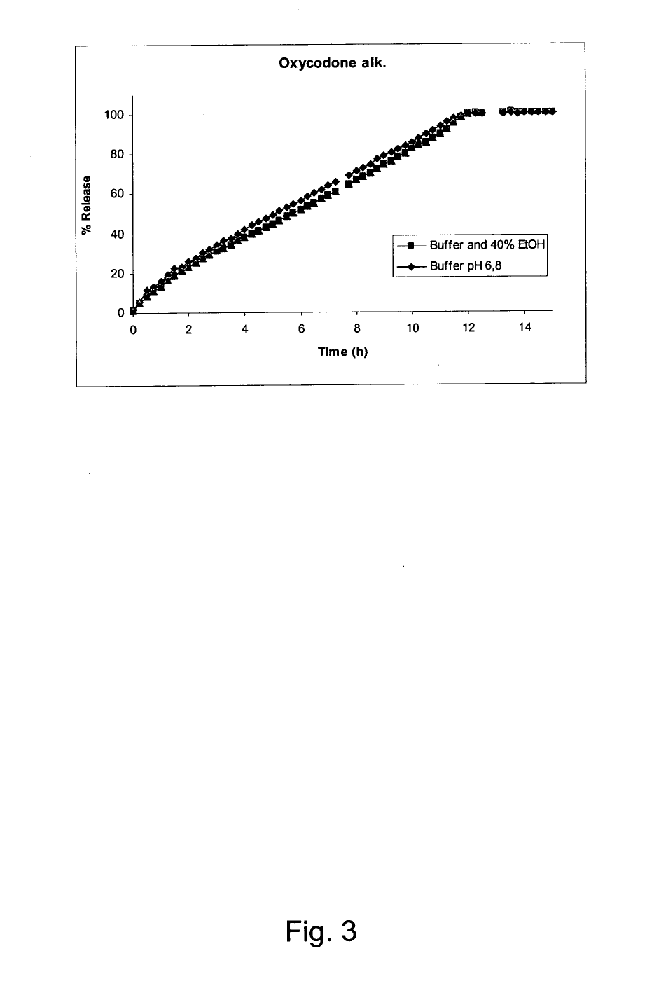

FIG. 3 shows in vitro dissolution of Oxycodone alkaloid, batch No. MMJ-0134-053, from Example 2

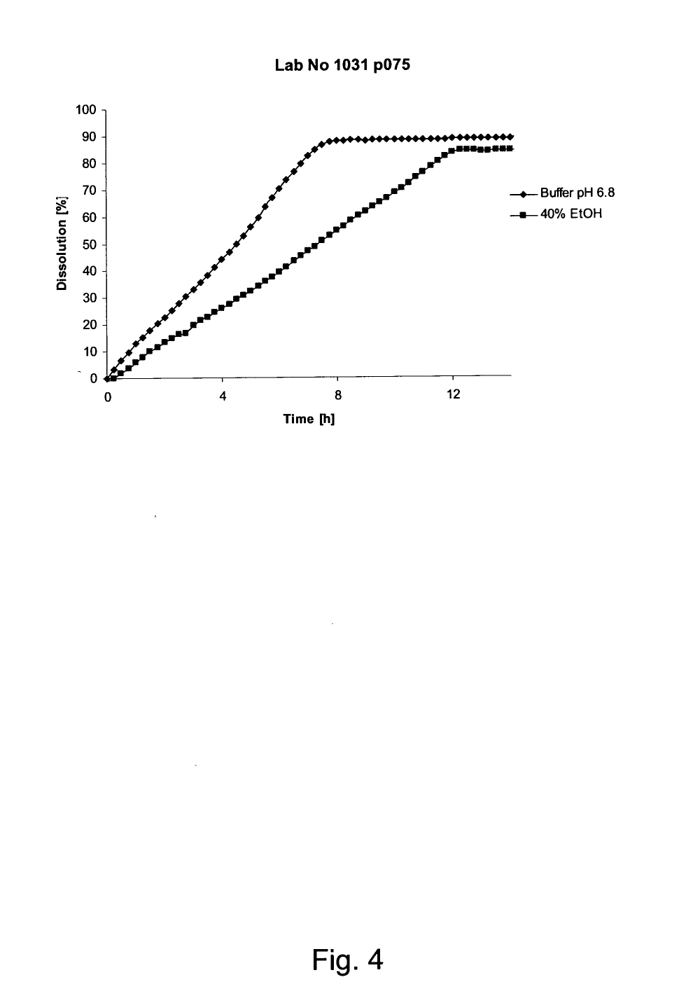

FIG. 4 shows in vitro dissolution of Hydrocodone bitartrate, batch No. 1031 p075 from Example 3

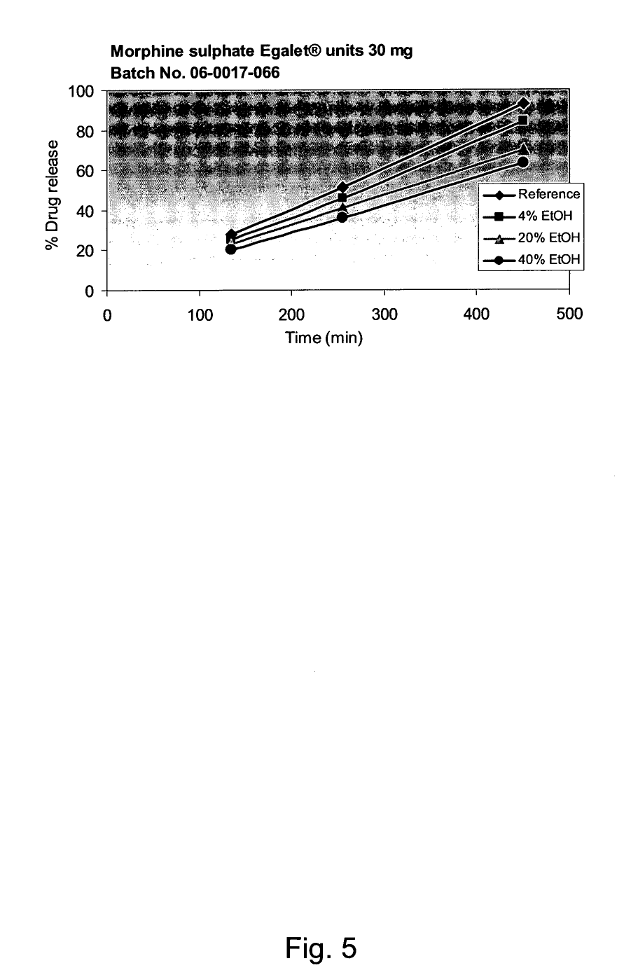

FIG. 5 shows in vitro dissolution of morphine sulphate, batch No. 06-0017-066, from Example 4

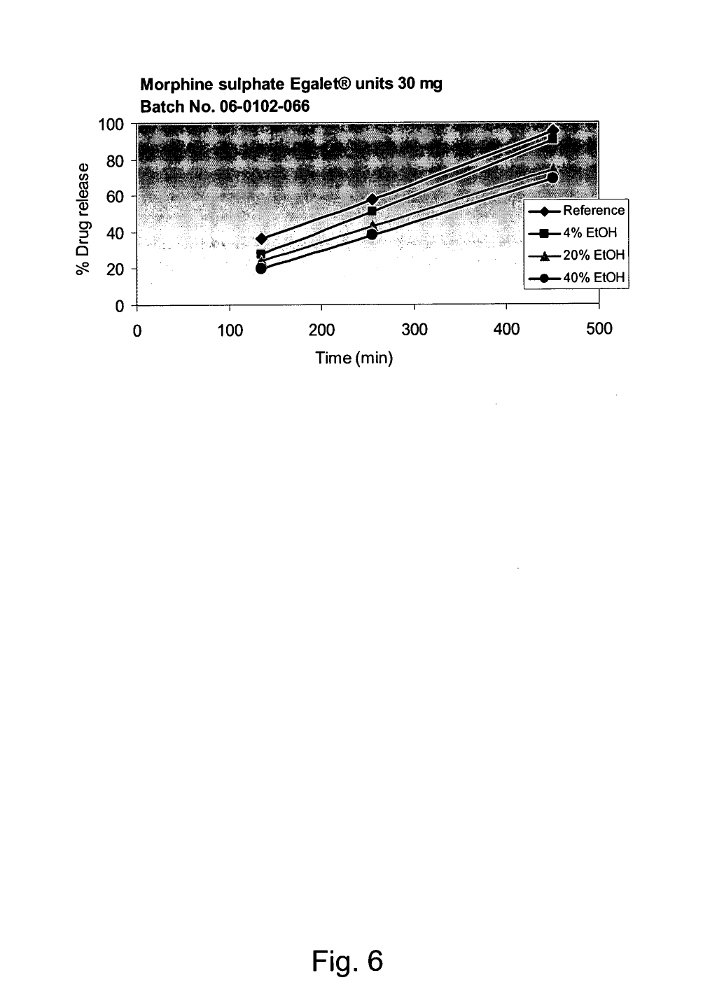

FIG. 6 shows in vitro dissolution of morphine sulphate, batch No. 06-0102-066, from Example 5

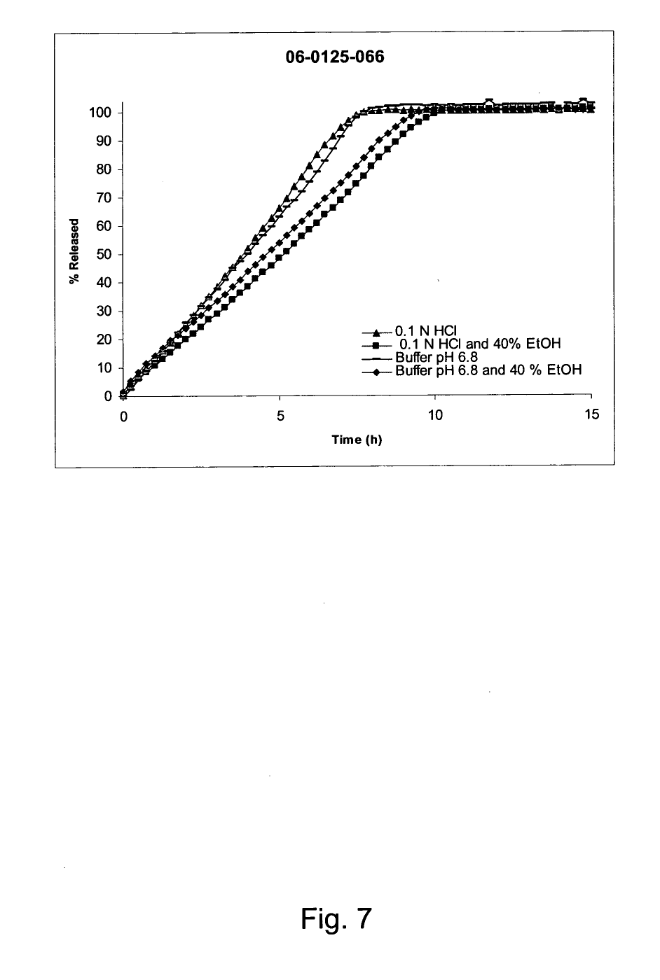

FIG. 7 shows in vitro dissolution of Morphine sulphate, batch No. 06-0125-066, from Example 6

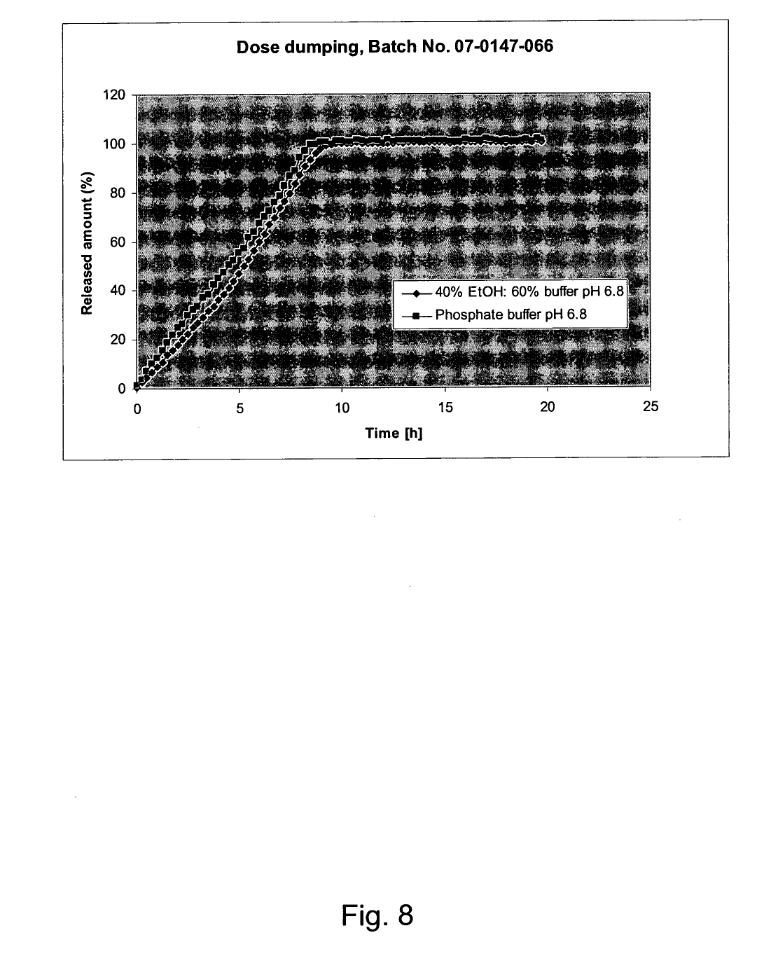

FIG. 8 shows in vitro dissolution of Morphine sulphate, batch No. 07-0147-066, from Example 7

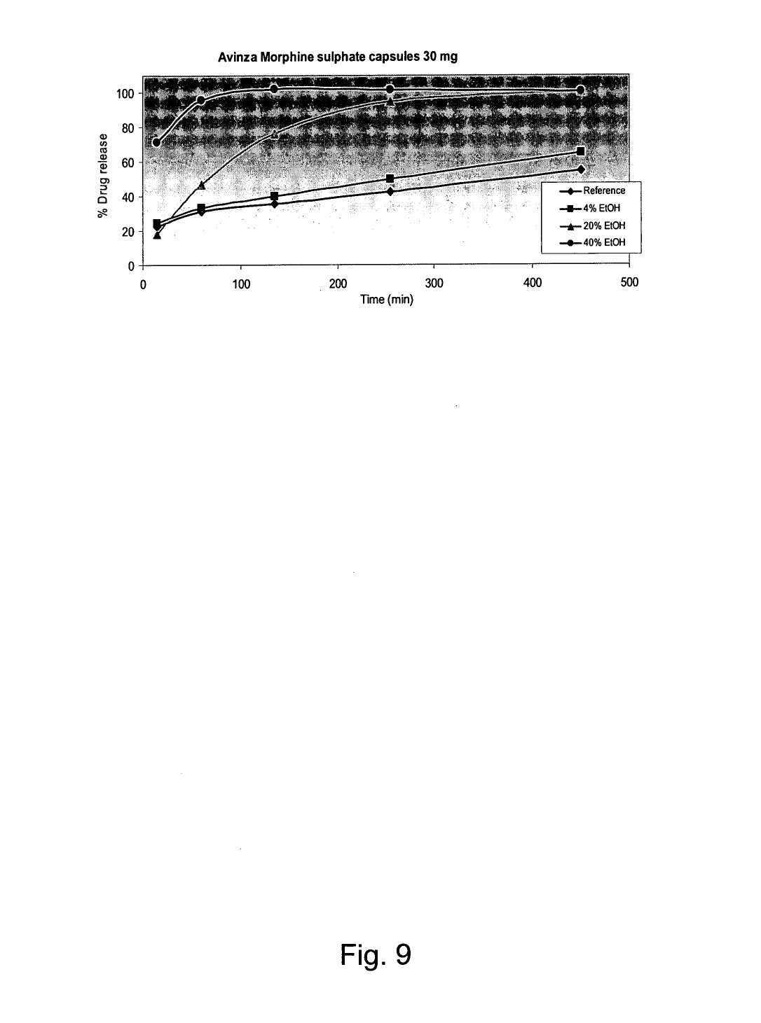

FIG. 9 shows in vitro dissolution of morphine sulphate, batch No. TCCH001, from Example 8,

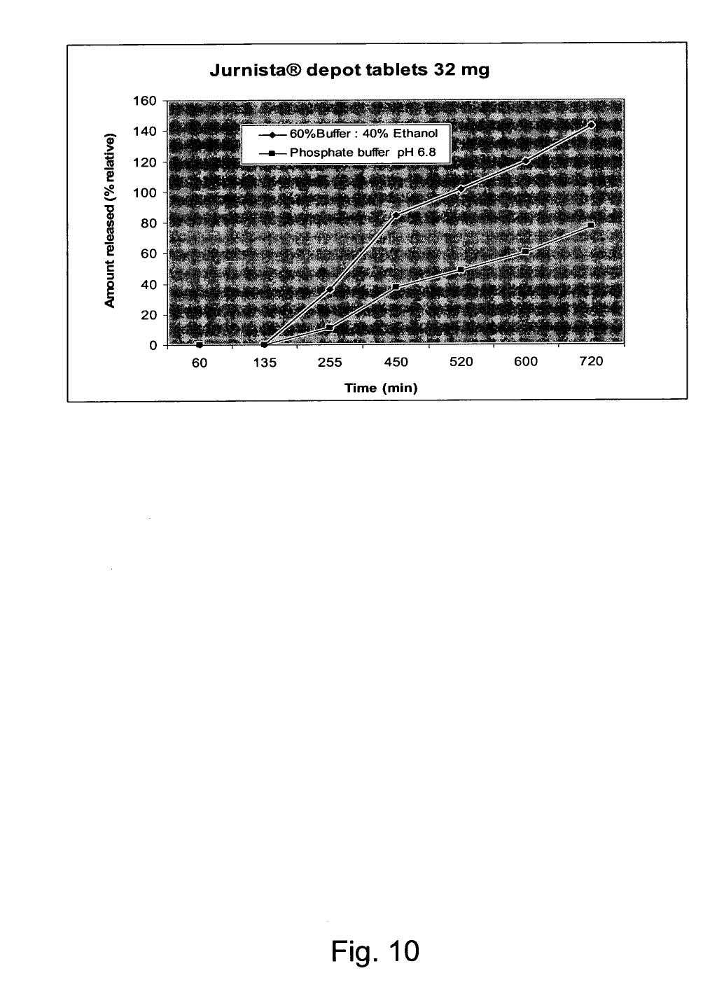

FIG. 10 shows in vitro dissolution of Hydromorphone Hydrochloride, batch No. 6JA945, from Example 9,

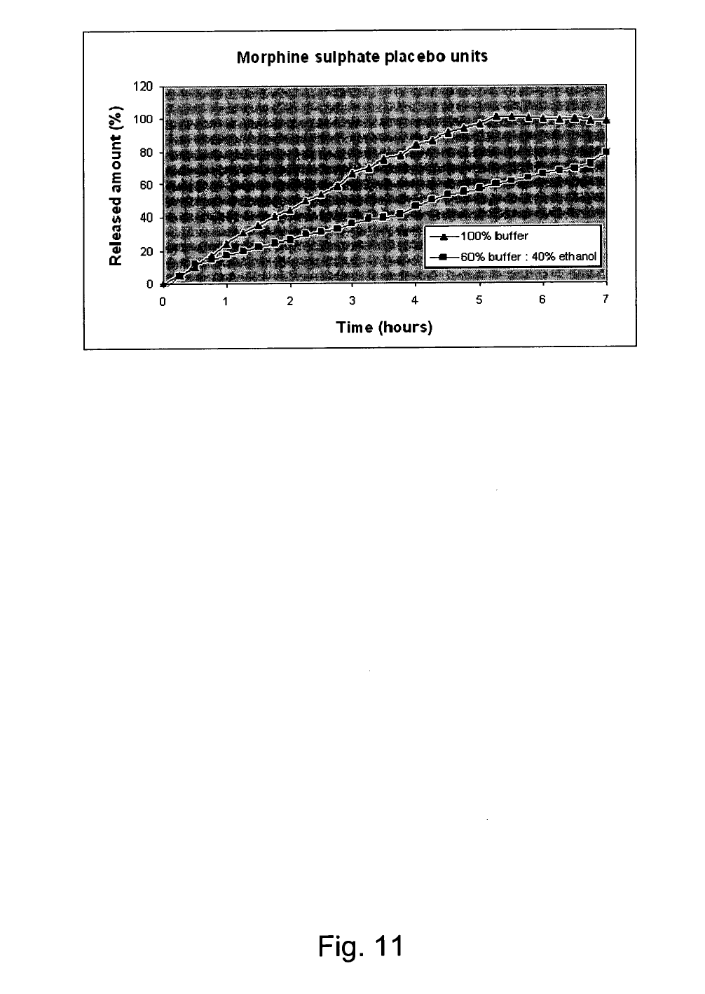

FIG. 11 shows in vitro dissolution of morphine sulphate placebo units, batch No. 03-0004-066, from Example 10,

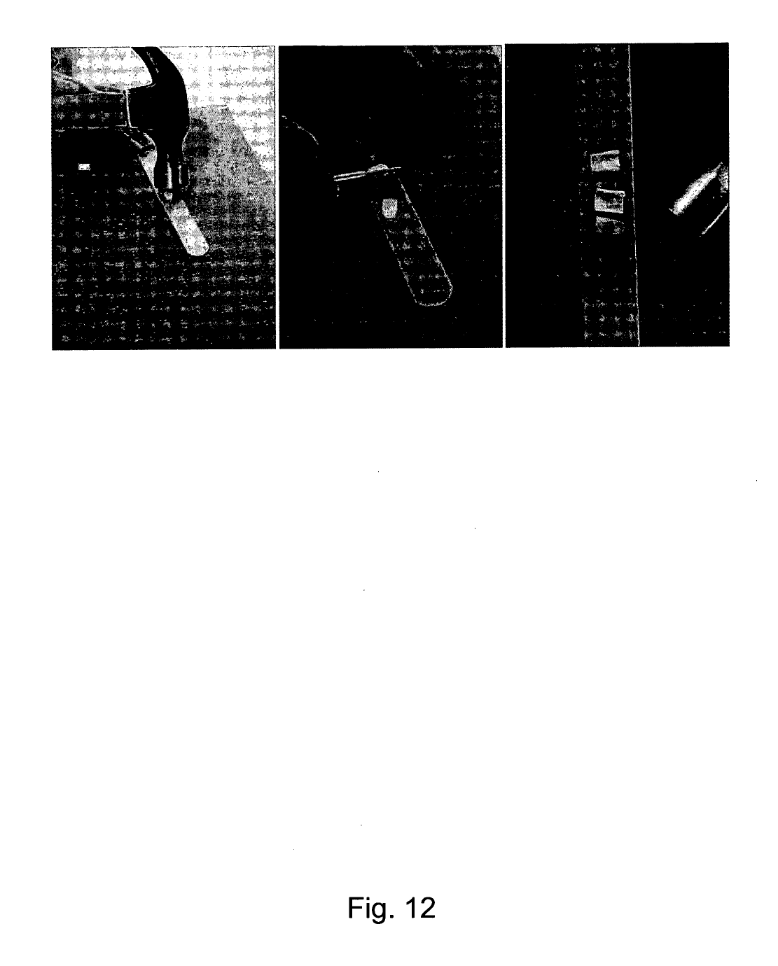

FIG. 12 A) shows the crushing of Egalet® Morphine Sulphate, B) the removal of the shell, and C) show the matrix and shell,

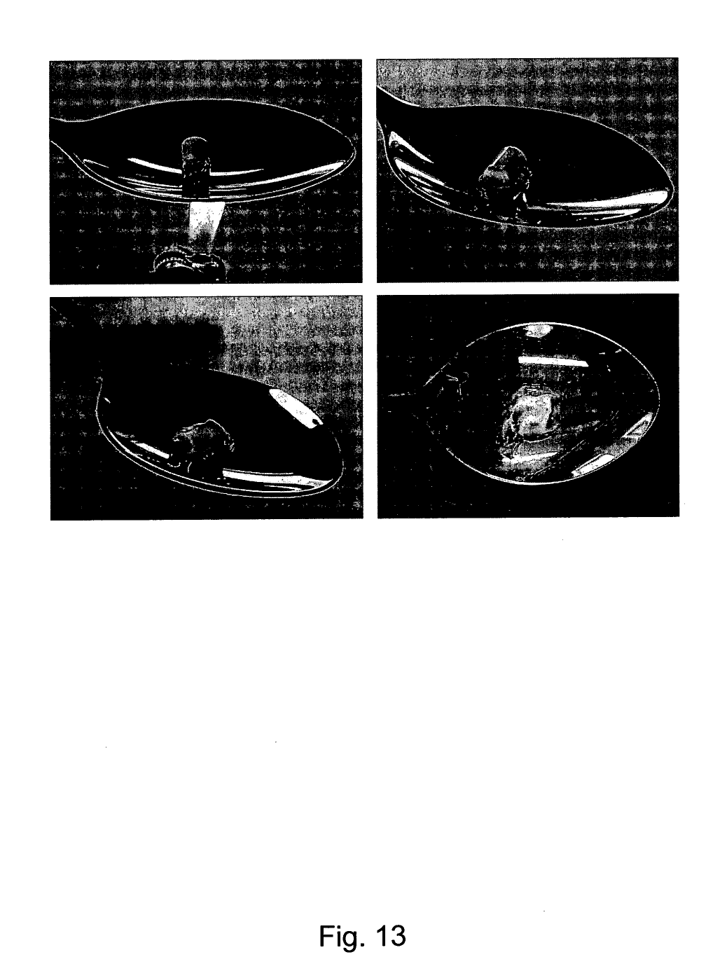

FIG. 13 shows the result of a melting test of Egalet® Morphine Sulphate, and

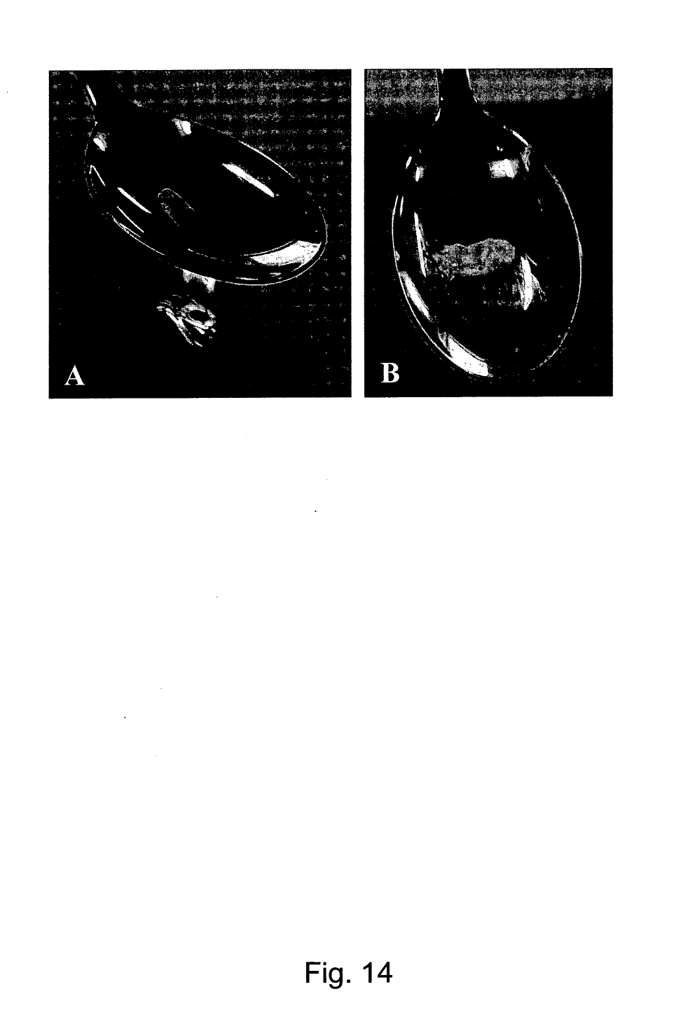

FIG. 14 shows the result of a melting test of Egalet® Morphine Sulphate.

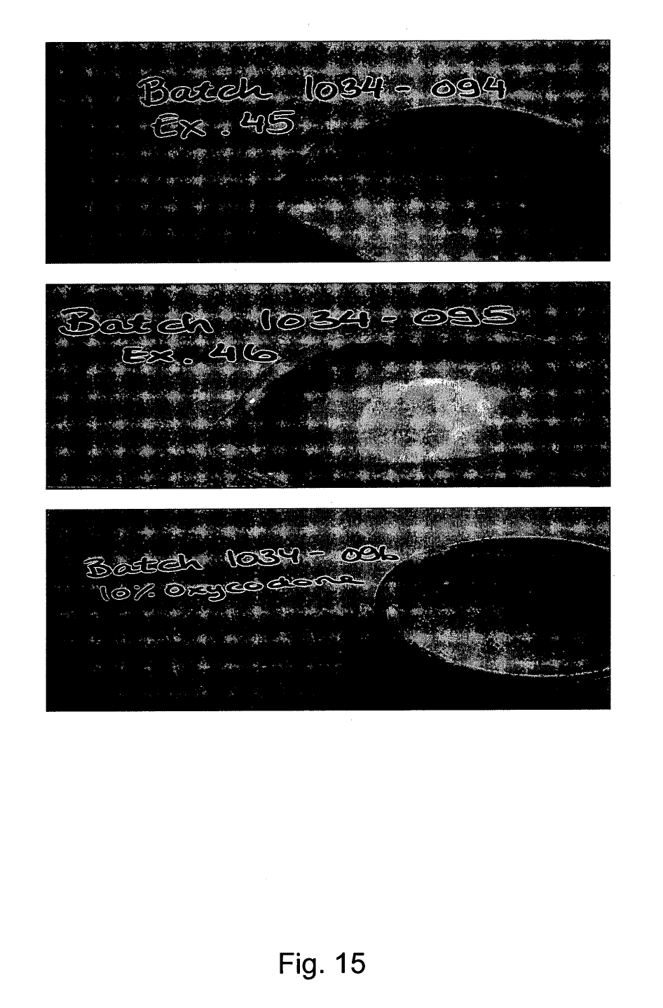

FIG. 15 shows the crushing of tablets (Batch No. 1034-094) from example 45 of WO 2006/058249, capsules (Batch No. 1034-095) from example 46 of WO 2006/058249 and capsules (Batch No. 1034-096) from example 2 of WO 2006/106344.

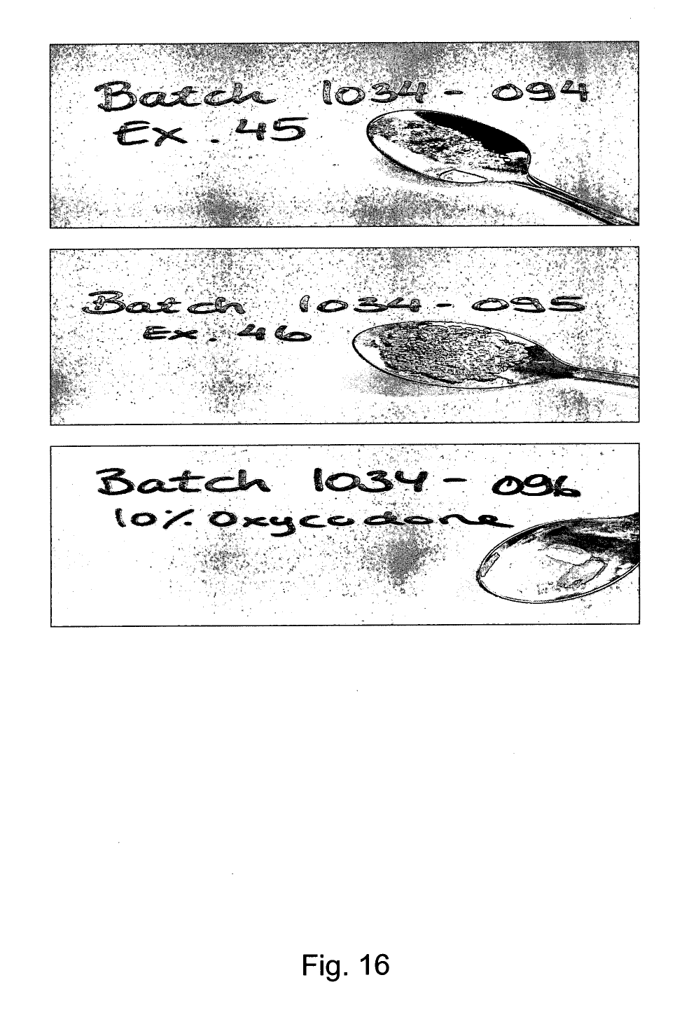

FIG. 16 shows the melting test of tablets (Batch No. 1034-094) from example 45 of WO 2006/058249, capsules (Batch No. 1034-095) from example 46 of WO 2006/058249 and capsules (Batch No. 1034-096) from example 2 of WO 2006/106344.

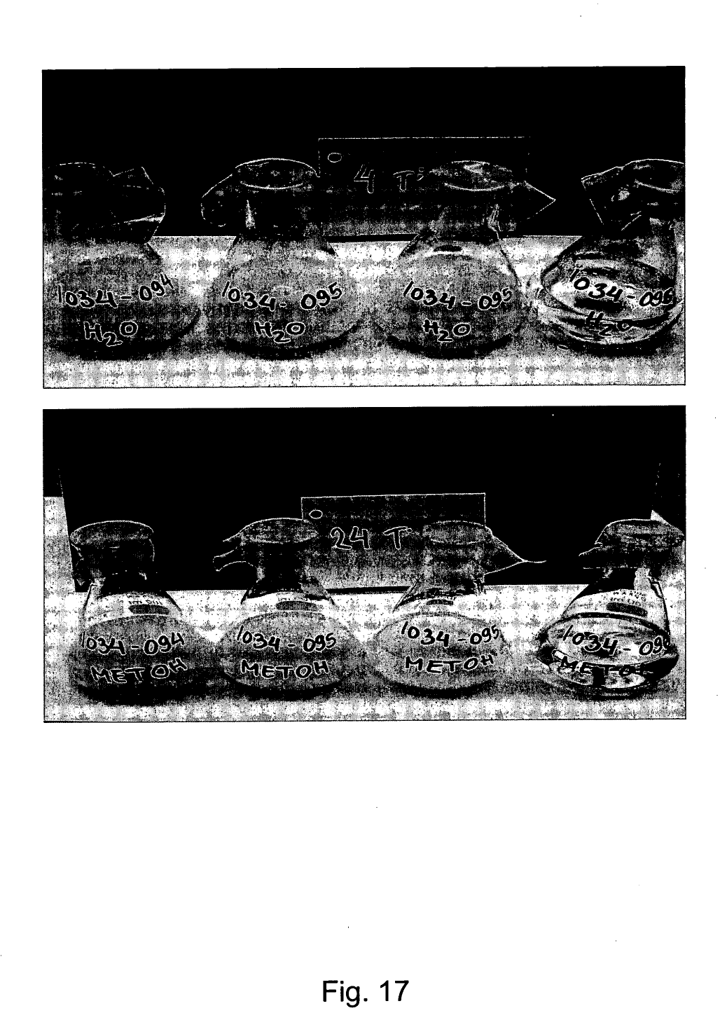

FIG. 17 shows the results from the extraction test after 4 and 24 hours of stirring tablets (Batch No. 1034-094) from example 45 of WO 2006/058249, capsules (Batch No. 1034-095) from example 46 of WO 2006/058249 and capsules (Batch No. 1034-096) from example 2 of WO 2006/106344.

### EXAMPLES

### Dissolution Test

Dissolution tests were performed in accordance with USP 30, NF 25, (711), Apparatus 2 (paddle method). The dissolution medium consisted either of dilute hydrochloric acid and/or ethanol or phosphate buffer solution pH 6.8 and/or ethanol. The volume of the dissolution medium was 900 ml and the rotation speed of the paddles was 50 rpm throughout the dissolution run. Samples were withdrawn at suitable time intervals and analysed for content of active drug substance by means of UV-detector or HPLC with UV-detector at a wavelength of 285 nm.

### Methods

A general method for the preparation of a controlled release composition is described below.

### Preparation of a Pharmaceutical Composition

An accurate amount of the polymer (i.e. in the examples below: polymer) is loaded into a MTI mixer followed by an accurate amount of the active drug substance and of other pharmaceutically acceptable excipients(s), if any. The mixing is performed at 900-2000 rpm and at a time period up to 20 min. At the start of the mixing the temperature is about 19-21° C. and the final temperature of the mixture is about 30-50° C. The mixture is then allowed to cool to room temperature and is ready to be fed into an injection moulding machine.

### Preparation of the Coating Composition

The coating composition is prepared by first adding the ethylcellulose then cetostearyl alcohol, and finally the titanium dioxide to an MTI-Mixer at a temperature about 19-21° C. After mixing at around 1000 rpm the mixer is stopped when the temperature reaches 40-50° C. and the adhered material is manually incorporated into the mixture. The mixture is left to cool for about 10 minutes. The mixing is then finalized with a short high-speed mix in order to minimize lump formation. The mixture is then allowed to cool to room temperature, after which it had a suitable consistency for being fed into an injection moulding machine.

### Example of Coat Composition

### Batch: 058-050-07-023B-A

| | | | Amount | | |
|---|---|---|---|---|---|
| % | Batch | Material | (g) | Weight (g) | step |
| 87 | 060601-C5C | Ethocel | 1218 | 1218.0 | 1 |
| 12 | 030120-A | Cetostearyl Alcohol | 168 | 168.0 | 2 |
| 11 | 030120-A | TiO2 | 14 | 14.0 | 3 |
| 100 | | Total | 1400 | 1400.0 | |

The final dosage units may be prepared according to two different methods. In one method, the coat and the matrix are moulded individually followed by a manually incorporation of the moulded matrix plug into the moulded coat. The injection moulding machine used is an Arburg Allrounder 220 S 250/60.

In the second method, the coat and matrix are moulded in one process where the coat is moulded in a first step and the matrix is moulded directly into the coat in a second step (co-moulding or 2 component moulding). The injection moulding machine used is Arburg Allrounder 420 V 800-60/35.

### Small Scale Preparation of a Pharmaceutical Composition

A mixture may be prepared by simple volumetric mixing of the components. 3 g of the mixture is then feeded into a table top injection molding machine (Haake MiniJet II, Thermo Electron, Karlsruhe, Germany) and molded directly into a pre-molden shell. Typical settings in the MiniJet are: Temperature 90-120° C. and pressure 600-800 bar.

### Example 1

### Preparation of Oxycodone Hydrochloride Containing Controlled Release Composition for Use According to the Invention

A composition (batch No. 07-0130-114) according to the invention was prepared form the following ingredients:

| | % (w/w) |
|---|---|
| Matrix | |
| Oxycodone hydrochloride | 50 |
| PEO 200 000 | 20 |
| PEO 300 000 | 25 |
| Poloxamer 338 | 5 |
| Shell | |
| Ethylcellulose | 87 |
| Ceto stearyl alchol | 12 |
| Titanium dioxide | 1 |

The composition was prepared as described above. The composition was 7.5 mm long, of cylindrical shape and with oval end surfaces.

The content of Oxycodone hydrochloride in the formulation is 160 mg.

The composition was subjected to the dissolution test described above. In addition to phosphate buffer medium and dilute hydrochloric acid, testing was performed in medium containing phosphate buffer pH 6.8.and ethanol at the ratio 60:40 (v/v) and dilute hydrochloric acid and ethanol at the ratio 60:40 (v/v). The following results were obtained:

% w/w release of Oxycodone
hydrochloride from the composition

| Time | | Buffer:Ethanol | | HCl:Ethanol |
|---|---|---|---|---|
| (minutes) | Buffer | 60:40 | HCl | 60:40 |
| 135 | 37 | 27 | 35 | 28 |
| 255 | 71 | 51 | 66 | 54 |
| 450 | 99 | 94 | 98 | 86 |

| | t_{50% w/w} | t_{50% w/w} | | t_{50% w/w} | t_{50% w/w} |
|---|---|---|---|---|---|
| Ratio | (Buffer)/ | (Buffer:Ethanol) | Ratio | (HCl)/ | (HCl:Ethanol) |
| (R₅₀) | hours | 60:40/hours | (R₅₀) | hours | 60:40/hours |
| 1.42 | 3.0 | 4.25 | 1.33 | 3.0 | 4.0 |

The results are also shown in FIGS. 1 & 2 and the release corresponds to a zero order release.

### Example 2

### Preparation of Oxycodone Alkaloid Containing Controlled Release Composition for Use According to the Invention

A composition (batch No. MMJ-0134-053) according to the invention was prepared form the following ingredients:

| | % (w/w) |
|---|---|
| Matrix | |
| Oxycodone alkaloid | 10 |
| Citric acid (CA) | 7 |
| Mannitol | 5 |
| PoloXamer 407 | 39 |
| PEO 300 000 | 17 |
| PEO 100 000 | 22 |
| Shell | |
| Ethylcellulose | 87 |
| Ceto stearyl alchol | 12 |
| Titanium dioxide | 1 |

The coating and the matrix were prepared as described above. The composition was 9 mm long, of cylindrical shape and with oval end surfaces.

The content of Oxycodone alkaloid in the formulation is 20 mg.

The composition was subjected to the dissolution test described above. In addition to phosphate buffer medium, testing was performed in medium containing phosphate buffer pH 6.8.and ethanol at the ratio 60:40 (v/v). The following results were obtained:

| | % w/w release of Oxycodone alkaloid from the composition | |
|---|---|---|
| Time | | Buffer:Ethanol |
| (minutes) | Buffer | (60:40) |
| 135 | 28 | 25 |
| 255 | 44 | 40 |
| 450 | 68 | 64 |

| | | t_{50% w/w} |
|---|---|---|
| Ratio | t_{50% w/w} | (Buffer:Ethanol) |
| (R₅₀) | (Buffer)/hours | 60:40/hours |
| 1.13 | 5.1 | 5.75 |

The results are shown in FIG. 3 and the release corresponds to a zero order release.

### Example 3

### Preparation of Hydrocodone Bitartrate Containing Controlled Release Composition for Use According to the Invention

A composition (Lab No 1031 p075) according to the invention was prepared form the following ingredients:

| | % (w/w) |
|---|---|
| Matrix | |
| Hydrocodone Bitartrate | 50 |
| PEO 300 000 | 25 |
| PEO 200 000 | 20 |
| Poloxamer 188 | 5 |
| Shell | |
| Ethylcellulose | 87 |
| Ceto stearyl alchol | 12 |
| Titanium dioxide | 1 |

The composition was prepared as described above. The composition was 9 mm long, of cylindrical shape and with circular end surfaces.

The content of Hydrocodone bitartrate in the formulation is 90 mg.

The composition was subjected to the dissolution test described above. In addition to phosphate buffer medium, testing was performed in medium containing phosphate buffer pH 6.8.and ethanol at the ratio 60:40 (v/v). The following results were obtained:

| | % release of Hydrocodone bitartrate from the composition | |
|---|---|---|
| Time | | Buffer:Ethanol |
| (minutes) | Buffer | (60:40) |
| 135 | 25 | 15 |
| 255 | 47 | 28 |
| 450 | 87 | 51 |

| | | t_{50% w/w} |
|---|---|---|
| Ratio | t_{50% w/w} | (Buffer:Ethanol) |
| (R₅₀) | (Buffer)/hours | 60:40/hours |
| 1.64 | 4.5 | 7.4 |

The results are also shown in FIG. 4 and the release corresponds to a zero order release.

### Example 4

### Preparation of a Morphine Sulphate Containing Controlled Release Composition for use according to the invention

A composition (batch No. 01-0017-066) according to the invention was prepared from the following ingredients:

| | % (w/w) |
|---|---|
| Matrix | |
| PEO 200 000 NF | 71.44 |
| Mophine sulphate | 15.96 |
| pentahydrate | |
| TPGS | 2.6 |
| Mannitol | 10.0 |
| Shell | |
| Ethylcellulose | 79.00 |
| Cetostearyl alcohol | 20.00 |
| Titanium dioxide | 1.00 |

The coating and the matrix were prepared as described above. The composition was 9 mm long, of cylindrical shape and with circular end surfaces.

The content of morphine sulphate in the composition corresponds to 30 mg morphine sulphate pentahydrate.

The composition was subjected to the dissolution test described above. In addition to phosphate buffer medium, testing was performed in medium containing phosphate buffer pH 6.8.and ethanol in various concentrations (v/v). The following results were obtained:

| | % w/w release of morphine sulphate from the composition | | | |
|---|---|---|---|---|
| Time | | Buffer:Ethanol | Buffer:Ethanol | Buffer:Ethanol |
| (minutes) | Buffer | (96:4) | (80:20) | (60:40) |
| 135 | 28 | 25 | 23 | 20 |
| 255 | 52 | 46 | 41 | 36 |
| 450 | 93 | 84 | 70 | 63 |

| | | t_{50% w/w} |
|---|---|---|
| Ratio | t_{50% w/w} | (Buffer:Ethanol) |
| (R₅₀) | (Buffer)/hours | 60:40/hours |
| 1.44 | 4.1 | 5.9 |

The results are shown in FIG. 5 and the release corresponds to a zero order release.

### Example 5

### Preparation of a Morphine Sulphate Containing Controlled Release Composition according to the invention

A composition (batch No. 06-0102-066) according to the invention was prepared from the following ingredients:

| | % (w/w) |
|---|---|
| Matrix | |
| PEO 200 000 NF | 71.44 |
| Mophine sulphate | 15.96 |
| pentahydrate | |
| TPGS | 2.6 |
| Mannitol | 10.0 |
| Shell | |
| Ethylcellulose | 79.00 |
| Cetostearyl alcohol | 20.00 |
| Titanium dioxide | 1.00 |

The coating and the matrix were prepared as described above. The composition was 9 mm long, of cylindrical shape and with circular end surfaces.

The content of morphine sulphate in the composition corresponds to 30 mg morphine sulphate pentahydrate.

The composition was subjected to the dissolution test described above. In addition to phosphate buffer medium, testing was performed in medium containing phosphate buffer pH 6.8.and ethanol in various concentrations (v/v). The following results were obtained:

| | % w/w release of morphine sulphate from the composition | | | |
|---|---|---|---|---|
| Time | | Buffer:Ethanol | Buffer:Ethanol | Buffer:Ethanol |
| (minutes) | Buffer | (96:4) | (80:20) | (60:40) |
| 135 | 36 | 28 | 24 | 20 |
| 255 | 57 | 51 | 43 | 38 |
| 450 | 95 | 90 | 74 | 68 |

| | | t_{50% w/w} |
|---|---|---|
| Ratio | t_{50% w/w} | (Buffer:Ethanol) |
| (R₅₀) | (Buffer)/hours | 60:40/hours |
| 1.51 | 3.7 | 5.6 |

The results are also shown in FIG. 6 and the release corresponds to a zero order release.

### Example 6

### Preparation of a Morphine Sulphate Containing Controlled Release Composition for Use According to the Invention

A composition (batch No. 06-0125-066) according to the invention was prepared from the following ingredients.

| | % (w/w) |
|---|---|
| Matrix | |
| PEO 200 000 NF | 71.44 |
| Morphine sulphate | 15.96 |
| Pentahydrate | |
| Mannitol | 10.0 |
| TPGS | 2.6 |
| Shell | |
| Ethylcellulose | 89.00 |
| Cetrostearyl alcohol | 10.00 |
| Titanium dioxide | 1.00 |

The coating and the matrix were prepared as described above. The composition was 9 mm long, of cylindrical shape and with circular end surfaces.

The content of morphine sulphate in the composition corresponds to 30 mg morphine sulphate Pentahydrate.

The composition was subjected to the dissolution test described above. In addition to phosphate buffer medium and dilute hydrochloric acid, testing was performed in medium containing phosphate buffer pH 6.8 and ethanol at the ratio 60:40 (v/v) and dilute hydrochloric acid and ethanol at the ratio 60:40 (v/v). The following results were obtained:

| | % release of morphine sulphate from the composition | | | |
|---|---|---|---|---|
| | | 0.1 N HCl: | | |
| Time | 0.1 N HCl | Ethanol | Buffer pH | Buffer pH 6.8: |
| (minutes) | pH 1 | 60:40 | 6.8 | Ethanol |
| 135 | 28.5 | 22.0 | 24.0 | 24.0 |
| 255 | 55.7 | 41.2 | 53.5 | 46.3 |
| 450 | 98.9 | 74.7 | 98.2 | 80.7 |

| | t_{50% w/w} | t_{50% w/w} | | t_{50% w/w} | t_{50% w/w} |
|---|---|---|---|---|---|
| Ratio | (HCl)/ | (HCl:Ethanol) | Ratio | (Buffer)/ | (Buffer:Ethanol) |
| (R₅₀) | hours | 60:40/hours | (R₅₀) | hours | 60:40/hours |
| 1.37 | 3.8 | 5.2 | 1.15 | 4.0 | 4.6 |

The results are shown in FIG. 7 and the release corresponds to a zero order.

### Example 7

### Preparation of a Morphine Sulphate Containing Controlled Release Composition According to the Invention

A composition (batch no. 07-0147-066) according to the invention was prepared from the following ingredients:

| | % (w/w) |
|---|---|
| Matrix | |
| PEO 300.000 | 35 |
| Morphine sulphate | 53 |
| pentahydrate | |
| Poloxamer 188 | 9 |
| Mannitol | 3 |
| Shell | |
| Ethylcellulose | 87.00 |
| Cetostearyl alcohol | 12.00 |
| Titanium dioxide | 1.00 |

The coating and the matrix were prepared as described above. The composition was 7.5 mm long, of cylindrical shape and with oval end surfaces.

The content of morphine sulphate in the composition corresponds to 100 mg morphine sulphate pentahydrate.

The composition was subjected to the dissolution test described above. In addition to phosphate buffer medium, testing was performed in medium containing phosphate buffer pH 6.8 and ethanol at the ratio 60:40 (v/v). The following results were obtained:

| | % w/w release of morphine sulphate from the composition | |
|---|---|---|
| Time | | Buffer:Ethanol |
| (minutes) | Buffer | (60:40) |
| 135 | 27 | 20 |
| 255 | 47 | 39 |
| 450 | 87 | 80 |

| | | t_{50% w/w} |
|---|---|---|
| Ratio | t_{50% w/w} | (Buffer:Ethanol) |
| (R₅₀) | (Buffer)/hours | 60:40/hours |
| 1.17 | 4.5 | 5.25 |

The results are shown in FIG. 8 and the release corresponds to a zero order release.

### Example 8

### Reference Example

Avinza Morphine Sulphate Extended-release Capsules 30 mg from King Pharmaceutical

The composition (batch No. TCCH001) contains both immediate release and extended release beads of morphine sulphate for once daily oral administration. SODAS® (Spheroidal Oral Drug Absorption System) technology is applied. Each capsule contains morphine sulphate, ammoniomethacrylate copolymers, fumaric acid, povidone, sodium lauryl sulphate, sugar starch spheres and talc.

Warning (information from prescribing information contained in the drug package) Avinza capsules are to be swallowed whole or the contents of the capsules sprinkled on applesauce. The capsule beads are not to be chewed, crushed or dissolved due to the risk of rapid release and absorption of a potentially fatal dose of morphine. Patients must not consume alcoholic beverages while on Avinza Therapy. Additionally, patients must not use prescription or non-prescription medications containing alcohol while on Avinza Therapy. Consumption of alcohol while taking Avinza may result in the rapid release and absorption of a potentially fatal dose of morphine.

The content of morphine sulphate in the composition corresponds to 30 mg morphine sulphate pentahydrate.

The composition was subjected to the dissolution test described above. In addition to phosphate buffer medium, testing was performed in medium containing phosphate buffer pH 6.8 and ethanol in various concentrations (v/v).The following results were obtained:

| | % w/w release of morphine sulphate from the composition | | | |
|---|---|---|---|---|
| Time | | Buffer:Ethanol | Buffer:Ethanol | Buffer:Ethanol |
| (minutes) | Buffer | (96:4) | (80:20) | (60:40) |
| 15 | 22.5 | 24.6 | 18.2 | 71.5 |
| 60 | 31.2 | 33.4 | 47.1 | 96.1 |
| 135 | 35.7 | 40.1 | 76.3 | 102.1 |
| 255 | 42.8 | 49.9 | 94.8 | 101.7 |
| 450 | 54.7 | 65.3 | 100.9 | 100.8 |

The results are shown in FIG. 9.

### Example 9

### Reference Example

### Jurnista Hydromorphone Hydrochloride Prolonged Release Tablets 32 mg from Janssen-Cilag

The composition is a depot tablet formulation (batch No. 6JA945) of hydromorphone hydrochloride for once daily oral administration, OROS® technology, employing osmosis, is applied. Each tablet contains hydromorphone hydrochloride, polyethylene oxide, povidone, magnesium stearate, yellow ferric oxide, butylhydroxytoluene, sodium chloride, hypromellose, black ferric oxide, lactose, cellulose acetate, macrogol and a coat which contains lactose, hypromellose, titanium dioxide, glycerol triacetate, red ferric oxide, yellow ferric oxide, indigo carmine and macrogol.

Warning (Information Derived from Prescribing Information Contained in the Drug package)

Jurnista tablets are to be swallowed whole. The tablets are not to be chewed, crushed or dissolved due to the risk of rapid release and absorption of a potentially fatal dose of hydromorphone. Patients must not consume alcoholic beverages while on Jurnista Therapy.

The content of hydromorphone hydrochloride in the composition corresponds to 32 mg of hydromorphone hydrochloride.

The composition was subjected to the dissolution test described above. In addition to phosphate buffer medium, testing was performed in medium containing phosphate buffer pH 6.8 and ethanol at the ratio 60:40 (v/v). The following results were obtained:

% w/w release of hydromorphone
hydrochloride from the composition

| Time | | Buffer:Ethanol |
|---|---|---|
| (minutes) | Buffer | (60:40) |
| 60 | 0.0 | 0.0 |
| 135 | 0.0 | 0.0 |
| 255 | 10.9 | 35.9 |
| 450 | 37.6 | 84.5 |
| 520 | 48.6 | 101.9 |
| 600 | 60.2 | 119.5 |
| 720 | 77.4 | 142.9 |

The results are shown in FIG. 10.

### Example 10

### Preparation of a Placebo Controlled Release Composition According to the Invention

A composition (batch No. 03-0004-066) according to the invention was prepared from the following ingredients:

| | % (w/w) |
|---|---|
| Matrix | |
| Mannitol | 12.1% |
| TPGS | 3.2% |
| PEO 200.000 | 84.7% |
| Shell | |
| Ethylcellulose | 79% |
| Cetostearyl alcohol | 20% |
| Titanium dioxide | 1% |

The coating and the matrix were prepared as described above. The composition was 9 mm long, of cylindrical shape and with circular end surfaces.

The composition was subjected to the dissolution test described above. In addition to phosphate buffer medium, testing was performed in medium containing buffer and ethanol at the ratio 60:40 (v/v). The content of Polyethylene oxide was analysed by means of UV-detector at a wavelength of 202 nm.

The results are also shown in FIG. 11 and the release corresponds to a zero order release.

### Example 11

### Resistance to Crushing of Compositions According to Any of Examples 1-7 and 10

Dosage units (i.e. formulations as exemplified in Examples 1-7 and 10 herein) were subjected to the crushing test described in Ph.Eur., which is a test intended to determine, under defined conditions, the resistance to crushing of tablets, measured by the force needed to disrupt them by crushing. An apparatus Pharmatest PTB311E was applied. The composition was placed between the jaws with the exposed matrix end Avisurfaces in line with the jaw. It was not possible to crush the 10 Egalet® dosage units. The upper limit of the apparatus applied is 300N i.e. the composition has hardness above 300N.

### Example 12

### Test for Abuse of Compositions According to the Invention

Egalet® dosage units are resistant towards abuse.

In the following, examples of different ways to extract Morphine sulphate from the dosage unit with the purpose of abuse are given. In connection with these examples it will be clarified that abuse of active drug substances with a high abuse potential is immensely difficult if not impossible when formulated in the Egalet® dosage unit. The dosage unit consists of a protective shell (coating) and the polymeric matrix, in which the active drug substance and other excipients are incorporated. The shell covers most of the matrix except for the ends. In the following examples the protective shell has been removed, so the given examples represent the “worst-case scenarios”.

### Chewing the Dosage Unit and Active Drug Substance Absorption of the Unit Through the Mouth Cavity

As the polymeric matrix unit is a somewhat hard polymer, it will require an unrealistic amount of water/saliva to soften the dosage unit sufficient to be chewable.

If it in some way has been possible to bite through the unit or somehow crush the unit absorption of the active drug substance through the mouth cavity will be associated with significant discomfort. Although active drug substance absorption may be faster through the mouth cavity, the dosage unit is designed to have a controlled release in the intestines after exposure to a very acidic environment (the stomach), so even if the unit is chewed the active drug substance will most likely not be released faster than if it was swallowed.

Solubilising the Dosage Unit in Different Solvents with the Purpose of Faster Active Drug Substance Release

An increment of the surface area exposed to the solvent would lead to a higher dissolution rate. So removing the shells and exposing the entire polymeric matrix surface area would give the highest possible dissolution rate.

It has been shown that even when a solvent, consisting of both Acetonitrile (highly hydrophobic) and Ammonium acetate (hydrophilic) is used, solubilisation of the dosage unit takes at least an hour when vigorously shaken. One should consider that Acetonitrile is not a usual remedy of the house hold.

It has been shown that solubilising the polymeric matrix in water (when stirred) of room-temperature takes at least 3.5 hours, while solubilisation of the matrix in water (stirred) of approximately 75° C. took about 1.5 hours. The chosen temperature should correspond to a hot cup of drinkable tea.

Addition of salts, which may enhance active drug substance solubility due to increased ion strength, may consequently give rise to a higher dissolution rate. It was seen that the polymeric matrix dissolved in a phosphate-buffer within 2-2.5 hours.

It may be attempted to solubilise the dosage unit in alcohol to obtain a larger effect, however it has been seen that the dissolution rate of the dosage unit in an alcohol-containing solution is considerably slower than in e.g. pure water (it took about 3 hours to solubilise the polymeric matrix in an ethanol-containing solution).

### Crushing the Dosage Unit

Another option would be to crush the unit, thereby extracting the active drug substance. Due to the plastic properties of the polymeric matrix, the matrix becomes flat when it is attempted crushed with a hammer, hence this procedure will not make the active drug substance more accessible. FIG. 12 illustrates an attempt to crush a coated matrix composition.

It was shown that a grinding mill indeed could pulverize the dosage unit and the matrix. It was seen that 8 grinded dosage units dissolved in pure tap water within half an hour and within 45 min in an alcohol-containing solution.

It was further seen that 8 grinded polymeric matrices dissolved within 15 min. in pure tap water and within 45 min in an alcohol-containing solution

The grinding mill, used in this experiment, is, however only accessible in the production industry and it is doubtful if an ordinary grinding mill for e.g. coffee would be forceful enough to crush the units. So crushing of de-shelled units in an ordinary household coffee mill was also attempted. It was possible to obtain a fine matrix ground from the first two units (although the yield was small), but with the third unit the machine broke down. It was concluded that such machines are not forceful enough to overcome the hardness combined with the plastic properties of the units. Hence the risk of abuse through grinding the units on ordinary household machines is regarded as minimal.

Melting the Unit with the Purpose of Extracting the Active Drug Substance

It has been seen that the texture of both the polymeric matrix as well as the dosage unit becomes very sticky when melted; hence the active drug substance will not be more accessible when melted (see FIGS. 13 and 14).

### Example 13

### Comparison Example

### Example 2 of WO 2006/106344

Capsules (batch No. 1034-096) from example 2 of WO 2006/106344 were prepared from the following ingredients:

| Composition | % (w/w) |
|---|---|
| Oxycodone hydrochloride | 10 |
| Poloxamer 188 | 90 |

Oxycodone hydrochloride was used as model drug substance instead of Temazepam.

Oxycodone hydrochloride and Poloxamer 188 was mixed and melted at 75° C. and filled in hard gelatine capsules.

### Crush Test

It was tried to crush the content of one capsule in a mortar and a fine white powder was obtained. The results from the crush test indicate that it is possible to obtain a fine powder for sniffing. The result is shown in FIG. 15.

### Melting Test

The content residue obtained from the crush test was placed on a metal spoon and a lighter was held under the spoon for at least 8 minutes. The fine white powder obtained in the crush test became a viscous fluid, which rapidly becomes more viscous at room temperature i.e. it was not injectable. The result is shown in FIG. 16.

### Extraction Test

Solvent extraction of oxycodone from the capsules were performed by dissolving the capsules in different solvents; water, ethanol and methanol. One unit was placed in 50 ml solvent with magnetic stirring throughout the whole experimental period. The samples were checked after 0, 1, 2, 3, 4 and 24 hours and the clarity was registered. The formulation tested could be abused via extraction in methanol, ethanol or water. The result is shown in FIG. 17 and the table below.

| | |
|---|---|
| Time | Solvent |

| (hours) | Water | Ethanol | Methanol |
|---|---|---|---|
| 0 | Unclear | Unclear | Unclear |
| 1 | Clear | Clear | Clear |
| 2 | Clear | Clear | Clear |
| 3 | Clear | Clear | Clear |
| 4* | Clear | Clear | Clear |
| 24* | Clear | Clear | Clear |

### Example 14

### Comparison Example

### Example 45 of WO 2006/058249

Tablets (batch No. 1034-094) from example 45 of WO 2006/058249 were prepared from the following ingredients:

| Composition | % (w/w) |
|---|---|
| Oxycodone hydrochloride | 4.1 |
| Niacin | 24.5 |
| Sodium lauryl sulphate | 1.4 |
| Microcystalline cellulose (part 1) | 12.2 |
| Povidone | 1.1 |
| Eudragit RS 30D (dry wt.) | 2.0 |
| Triacetin | 0.4 |
| Stearyl alcohol | 5.1 |
| Microcystalline cellulose (part 2) | 33.1 |
| Polyethylene oxide | 5.1 |
| Crospovidone | 10.2 |
| Talc | 0.5 |
| Magnesium stearate | 0.3 |

Eudragit RS 30D and Triacetin were plasticizing by mixing it to a homogeneous mixture. Next Oxycodone hydrochloride, Niacin, Sodium lauryl sulphate, Microcystalline cellulose and Povidone were added and further mixed. Stearyl alcohol was melted and combined to the mixture to achieve waxed granulates. The waxed granulate was blended with additional Microcystalline cellulose, Polyethylene oxide, Crospovidone, Talc and Magnesium stearate. The resulting composition was then compressed into tablets with a weight of approximately 490 mg.

10 round tablets were subjected to the crushing test described in Ph.Eur. An apparatus Pharmatest PTB311E was applied. The round tablets were measured one by one and the results are listed below.

| | Diameter | Hardness/N |
|---|---|---|
| Mean | 13.01 | 80.5 |
| Minimum | 12.99 | 72.0 |
| Maximum | 13.02 | 89.0 |

### Crush Test

It was tried to crush the content of one tablet in a mortar and a fine white powder was obtained. The results from the crush test indicate that it is possible to obtain a fine powder for sniffing. The result is shown in FIG. 15.

### Melting Test

The content residue obtained from the crush test was placed on a metal spoon and a lighter was held under the spoon for at least 8 minutes. The fine white powder obtained in the crush test became burnt powder. The formulation is not suitable for injection. The result is shown in FIG. 16.

### Extraction Test

Solvent extraction of oxycodone from the tablets were performed by dissolving the tablets in different solvents; water, ethanol and methanol. One unit was placed in 50 ml solvent with magnetic stirring throughout the whole experimental period. The samples were checked after 0, 1, 2, 3, 4 and 24 hours and the clarity was registered. The formulation tested could not be abused via extraction in methanol, ethanol or water. The result is shown in FIG. 17 and the table below.

| | |
|---|---|
| Time | Solvent |

| (hours) | Water | Ethanol | Methanol |
|---|---|---|---|
| 0 | Unclear | Unclear | Unclear |
| 1 | Unclear | Unclear | Unclear |
| 2 | Unclear | Unclear | Unclear |
| 3 | Unclear | Unclear | Unclear |
| 4* | Unclear | Unclear | Unclear |
| 24* | Clear | Clear | Clear |

### Example 15

### Comparison Example

### Example 46 of WO 2006/058249

Capsules (batch No. 1034-095) from example 46 of WO 2006/058249 were prepared from the following ingredients:

| Composition | % (w/w) |
|---|---|
| Oxycodone hydrochloride | 3 |
| Niacin | 23 |
| Sodium lauryl sulphate | 1 |
| Eudragit RSPO | 14 |
| Ethylcellulose | 1 |
| Stearyl alcohol | 5 |
| Microcystalline cellulose | 37 |
| Polyethylene oxide | 7 |
| Crospovidone | 9 |

Oxycodone hydrochloride was used as model drug substance instead of Hydromorphone hydrochloride.

Stearyl alcohol flakes were milled and blended with Oxycodone hydrochloride, Niacin, Sodium lauryl sulphate, Eudragit RSPO, Ethylcellulose. The mixture was heated and processed into granulates. The granulate were blended with Microcrystalline cellulose, Polyethylene oxide and Crospovidone and filled in hard gelatine capsules.

### Crush Test

It was tried to crush the content of one capsule in a mortar and a fine white powder was obtained. The results from the crush test indicate that it is possible to obtain a fine powder for sniffing. The result is shown in FIG. 15.

### Melting Test

The content residue obtained from the crush test was placed on a metal spoon and a lighter was held under the spoon for at least 8 minutes. The fine white powder obtained in the crush test became burnt powder. The formulation is not suitable for injection. The result is shown in FIG. 16.

### Extraction Test

Solvent extraction of oxycodone from the capsules were performed by dissolving the capsules in different solvents; water, ethanol and methanol. One unit was placed in 50 ml solvent with magnetic stirring throughout the whole experimental period. The samples were checked after 0, 1, 2, 3, 4 and 24 hours and the clarity was registered. The formulation tested could not be abused via extraction in methanol, ethanol or water. The result is shown in FIG. 17 and the table below.

| | |
|---|---|
| Time | Solvent |

| (hours) | Water | Ethanol | Methanol |
|---|---|---|---|
| 0 | Unclear | Unclear | Unclear |
| 1 | Unclear | Unclear | Unclear |
| 2 | Unclear | Unclear | Unclear |
| 3 | Unclear | Unclear | Unclear |
| 4* | Unclear | Unclear | Unclear |
| 24* | Unclear | Unclear | Unclear |

1-26. (canceled)

27. A composition providing controlled release of one or more drug substances, the composition comprising:
a matrix comprising one or more drug substances and one or more polyglycols selected from polyethylene glycol and polyethylene oxides; and
a coating that is substantially insoluble, non-erodible and non-permeable to water, said coating partly covering the matrix;
wherein the composition is formulated such that the one or more drug substances are released from the composition by erosion of the matrix and said composition does not exhibit ethanol-induced dose dumping of the one or more active agents.

28. A composition according to claim 27, wherein one or both of the polyethylene glycol and the polyethylene oxide is a substantially water soluble crystalline or semi-crystalline polymer.

29. A composition according to claim 27, wherein the matrix further comprises a polyglycol co-polymer.

30. A composition according to claim 27, wherein the total concentration of polyglycols in the composition is selected from about 5% w/w to about 99.9% w/w, about 10% w/w to about 95% w/w, about 15% w/w to about 90% w/w, about 20% w/w to about 85% w/w, about 30% w/w to about 85% w/w, about 30% w/w to about 99% w/w, about 35% w/w to about 95% w/w, about 35% w/w to about 90% w/w, about 35% w/w to about 85% w/w, about 35% w/w to about 80% w/w, about 40% w/w to about 75% w/w, about 45% w/w to about 70% w/w, about 45% w/w to about 65% w/w, about 55% w/w to about 85% w/w, and about 60% w/w to about 85% w/w.

31. A composition according to claim 27, wherein one or both of the polyethylene glycol and the polyethylene oxide has a molecular weight of at least about 20,000.

32. A composition according to claim 27, wherein one or both of the polyethylene glycol and the polyethylene oxide has a molecular weight selected from about 20,000 daltons, about 20,000 to about 700,000 daltons, about 20,000 to about 600,000 daltons, about 35,000 to about 500,000 daltons, about 35,000 to about 400,000 daltons, about 35,000 to about 300,000 daltons, about 50,000 to about 300,000 daltons, about 35,000 daltons, about 50,000 daltons, about 75,000 daltons, about 100,000 daltons, about 150,000 daltons, about 200,000 daltons, about 250,000 daltons, about 300,000 daltons, and about 400,000 daltons.

33. A composition according to claim 27, wherein one or both of the polyethylene glycol and the polyethylene oxide has a molecular weight selected from about 35,000 daltons, about 50,000 daltons, about 100,000 daltons, about 200,000 daltons and about 300,000 daltons.

34. A composition according to claim 29, wherein the polyglycol co-polymer has a molecular weight of at least about 2,000 daltons.

35. A composition according to claim 34, wherein the polyglycol co-polymer is a polaxmer that has the formula HO(C₂H₄O)ₐ(C₃H₆O)_{b}(C₂H₄O)ₐH, and a is an integer selected from about 10 to about 150, from about 30 to about 140, from about 50 to about 100, from about 65 to about 90, and from about 70 to about 90, and b is an integer selected from about 10 to about 80, from about 15 to about 80, from about 20 to about 60, and from about 25 to about 55.

36. A composition according to claim 29, wherein the polyglycol co-polymer is a polaxmer that has a molecular weight selected from about 2,000 to about 30,000 daltons, from about 2,000 daltons to about 20,000 daltons, from about 4,000 daltons to about 18,000 daltons, and from about 6,000 daltons to about 10,000 daltons.

37. A composition according to claim 29, wherein the polyglycol co-polymer has a HLB value of at least about 18.

38. A composition according to claim 29, wherein the concentration of the polyglycol co-polymer in the pharmaceutical composition is selected from about 2.5% w/w to about 99.5% w/w, about 5% w/w to about 99.5% w/w, about 5% w/w to about 95% w/w, about 5% w/w to about 90% w/w, about 5% w/w to about 85% w/w, about 10% w/w to about 80% w/w, about 10% w/w to about 70% w/w, about 10% w/w to about 60% w/w, about 10% w/w to about 50% w/w, about 15% w/w to about 50% w/w, about 10% w/w to about 45% w/w, about 10% w/w to about 40% w/w, about 15% w/w to about 40% w/w, about 15% w/w to about 35% w/w, and about 15% w/w to about 30% w/w.

39. A composition according to claim 27, wherein the composition further exhibits a release rate of the one or more drug substances in ethanol that is equal to or lower than the release rate of the one or more drug substances exhibited in water.

40. A composition according to claim 27, wherein the composition further exhibits a release rate of the one or more drug substances in ethanol that is 1.5 times lower than the release rate of the one or more drug substances exhibited in water.

41. A composition according to claim 27, wherein the composition further exhibits a ratio (R₅₀) between t_{50% w/w}(40% ethanol in medium 1) and t_{50% w/w} (medium 1) that is 1 or more.

42. A composition according to claim 41, wherein the ratio R₅₀ is selected from at the most 5, at the most 4, at the most 3 and at the most 2.

43. A composition according to claim 41, wherein the ratio R₅₀ is selected from 1 to 1.5, from 1 to 1.4, from 1 to 1.3, from 1 to 1.2, from 1 to 1.1, from 1 to 1.05, and about 1.

44. A composition according to claim 27, wherein the composition is a solid dosage form.

45. A composition according to claim 27, wherein the composition is designed for oral administration.

46. A composition according to claim 45, wherein the composition is in the form of tablets, capsules or sachets.

47. A composition according to claim 27, wherein the pharmaceutical composition is an injection molded or extruded composition.

48. A composition according to claim 27, wherein the composition is compressed.

49. A composition according to claim 27, wherein the composition is resistant to crushing, whereby the composition is resistant to drug abuse.

50. A composition according to claim 27, wherein the composition is resistant to isolation of the drug substance by melting or ethanol extraction.

51. A composition according to claim 27, wherein the composition is resistant to isolation of the drug substance by crushing, melting or ethanol extraction.

52. A method for delivery of one or more drug substances to a subject in need thereof, the method comprising:
providing a composition for the controlled release of the one or more drug substances, the composition comprising,
a matrix comprising one or more drug substances and one or more polyglycols selected from polyethylene glycol and polyethylene oxides; and
a coating that is substantially insoluble, non-erodible and non-permeable to water, said coating partly covering the matrix;
wherein the composition is formulated such that the one or more drug substances are released from the composition by erosion of the matrix and said composition does not exhibit ethanol-induced dose dumping of the one or more active agents; and
administering said composition to the subject.

53. A method according to claim 52, wherein one or both of the polyethylene glycol and the polyethylene oxide included in the composition for controlled release is a substantially water soluble crystalline or semi-crystalline polymer.

54. A method according to claim 52, wherein the matrix included in the composition for controlled release further comprises a polyglycol co-polymer.

55. A method according to claim 52, wherein the total concentration of polyglycols in the composition for controlled release is selected from about 5% w/w to about 99.9% w/w, 10% w/w to about 95% w/w, about 15% w/w to about 90% w/w, about 20% w/w to about 85% w/w, about 30% w/w to about 85% w/w, about 30% w/w to about 99% w/w, about 35% w/w to about 95% w/w, about 35% w/w to about 90% w/w, about 35% w/w to about 85% w/w, about 35% w/w to about 80% w/w, about 40% w/w to about 75% w/w, about 45% w/w to about 70% w/w, about 45% w/w to about 65% w/w, about 55% w/w to about 85% w/w, and about 60% w/w to about 85% w/w.

56. A method according to claim 52, wherein one or both of the polyethylene glycol and the polyethylene oxide included in the composition for controlled release has a molecular weight of at least about 20,000.

57. A method according to claim 52, wherein one or both of the polyethylene glycol and the polyethylene oxide included in the composition for controlled release has a molecular weight selected from about 20,000 daltons, about 20,000 to about 700,000 daltons, about 20,000 to about 600,000 daltons, about 35,000 to about 500,000 daltons, about 35,000 to about 400,000 daltons, about 35,000 to about 300,000 daltons, about 50,000 to about 300,000 daltons, about 35,000 daltons, about 50,000 daltons, about 75,000 daltons, about 100,000 daltons, about 150,000 daltons, about 200,000 daltons, about 250,000 daltons, about 300,000 daltons, and about 400,000 daltons.

58. A method according to claim 52, wherein one or both of the polyethylene glycol and the polyethylene oxide included in the composition for controlled release has a molecular weight selected from about 35,000 daltons, about 50,000 daltons, about 100,000 daltons, about 200,000 daltons, and about 300,000 daltons.

59. A method according to claim 54, wherein the polyglycol co-polymer has a molecular weight of at least about 2,000 daltons.

60. A method according to claim 59, wherein the polyglycol co-polymer is a poloxamer that has the formula HO(C₂H₄O)ₐ(C₃H₆O)_{b}(C₂H₄O)ₐH, and a is an integer selected from about 10 to about 150, about 30 to about 140, about 50 to about 100, about 65 to about 90, about 70 to about 90, and b is an integer selected from about 10 to about 80, about 15 to about 80, about 20 to about 60, and about 25 to about 55.

61. A method according to claim 54, wherein the polyglycol copolymer is a poloxamer that has a molecular weight selected from about 2,000 daltons to about 30,000 daltons, about 2,000 daltons to about 20,000 daltons, about 4,000 daltons to about 18,000 daltons, and about 6,000 daltons to about 10,000 daltons.

62. A method according to claim 54, wherein the polyglycol co-polymer has a HLB value of at least about 18.

63. A method according to claim 54, wherein the concentration of the polyglycol co-polymer in the composition for controlled release is selected from about 2.5% w/w to about 99.5% w/w, about 5% w/w to about 99.5% w/w, about 5% w/w to about 95% w/w, about 5% w/w to about 90% w/w, about 5% w/w to about 85% w/w, about 10% w/w to about 80% w/w, about 10% w/w to about 70% w/w, about 10% w/w to about 60% w/w, about 10% w/w to about 50% w/w, about 15% w/w to about 50% w/w, about 10% w/w to about 45% w/w, about 10% w/w to about 40% w/w, about 15% w/w to about 40% w/w, about 15% w/w to about 35% w/w, and about 15% w/w to about 30% w/w.

64. A method according to claim 52, wherein the composition for controlled release further exhibits a release rate of the one or more drug substances in ethanol that is equal to or lower than the release rate of the one or more drug substances exhibited in water.

65. A method according to claim 52, wherein the composition for controlled release further exhibits a release rate of the one or more drug substances in ethanol that is 1.5 times lower than the release rate of the one or more drug substances exhibited in water.

66. A method according to claim 52, wherein the composition for controlled release further exhibits a ratio (R₅₀) between t_{50% w/w}(40% ethanol in medium 1) and t_{50% w/w} (medium 1) that is 1 or more.

67. A method according to claim 66, wherein the ratio R₅₀ is selected from at the most 5, at the most 4, at the most 3, and at the most 2.

68. A method according to claim 66, wherein the ratio R₅₀ is selected from 1 to 1.5, from 1 to 1.4, from 1 to 1.3, from 1 to 1.2, from 1 to 1.1, from 1 to 1.05, and about 1.

69. A method according to claim 52, wherein the composition for controlled release is a solid dosage form.

70. A method according to claim 52, wherein the composition for controlled release is designed for oral administration.

71. A method according to claim 52, wherein the composition for controlled release is in the form of tablets, capsules or sachets.

72. A method according to claim 52, wherein the composition for controlled release is an injection molded or extruded composition.

73. A method according to claim 52, wherein the composition for controlled release is compressed.

74. A method according to claim 52, wherein the composition for controlled release is resistant to crushing, whereby the composition is resistant to drug abuse.

75. A method according to claim 52, wherein the composition for controlled release is resistant to isolation of the drug substance by melting or ethanol extraction.

76. A method according to claim 52, wherein the composition for controlled release is resistant to isolation of the drug substance by crushing, melting or ethanol extraction.
